(19) United States
(12) Patent Application Publication
(10) Pub. No.: US 2011/0060183 A1
(43) Pub. Date: Mar. 10, 2011

(54) MULTI-INSTRUMENT ACCESS DEVICES AND SYSTEMS

(76) Inventors: Salvatore Castro, Raleigh, NC (US); Jeffrey A. Smith, Petaluma, CA (US); Geoffrey A. Orth, Sebastopol, CA (US)

(21) Appl. No.: 12/511,043

(22) Filed: Jul. 28, 2009

Related U.S. Application Data
(63) Continuation-in-part of application No. 12/209,408, filed on Sep. 12, 2008.
(60) Provisional application No. 61/153,644, filed on Feb. 19, 2009.
(60) Provisional application No. 61/159,805, filed on Mar. 13, 2009.
(60) Provisional application No. 60/971,903, filed on Sep. 12, 2007.

(51) Int. Cl. A61B 1/00

(52) U.S. Cl. 600/104

(57) ABSTRACT

A surgical access system for use in minimally invasive procedures such as single port or laparoscopic surgery. The system has a sealed base positionable in an incision formed in a body wall and at least two access tubes extending through the base. Each access tube includes a rigid tube having a fixed pre-formed shape including a bend in its distal section. The rigid tubes are restrained against pivotable movement relative to the base, but can be axially rotated and longitudinally repositioned relative to the base. A deflectable tubes extends from the distal end of each rigid tube. Each deflectable tube has a lumen for passage of a medical instrument, as well as a proximal actuator which engages a pullwire to deflect the tube when the user manipulates the instrument's handle.

This application claims the benefit of U.S. Provisional Application No. 61/153,644, filed Feb. 19, 2009, and U.S. Provisional Application No. 61/159,805, filed Mar. 13, 2009. This application is also a continuation-in-part of U.S. application Ser. No. 12/209,408, filed Sep. 12, 2008, which claims the benefit of U.S. Provisional Application No. 60/971,903, filed Sep. 12, 2007. Each of the aforementioned patent applications is incorporated herein by reference.

### TECHNICAL FIELD OF THE INVENTION

The present invention relates to the field of access devices through which medical instruments may be introduced into an incision or puncture opening formed in a body wall.

### BACKGROUND

Surgery in the abdominal cavity is frequently performed using open laparoscopic procedures, in which multiple small incisions or ports are formed through the skin and underlying muscle and peritoneal tissue to gain access to the peritoneal site using the various instruments and scopes needed to complete the procedure. The peritoneal cavity is typically inflated using insufflation gas to expand the cavity, thus improving visualization and working space. Further developments have lead to systems allowing such procedures to be performed using only a single port.

In single port surgery (“SPS”) procedures, it is useful to position a device within the incision to give sealed access to the operative space without loss of insufflation pressure. Ideally, such a device provides sealed access for multiple instruments while avoiding conflict between instruments during their simultaneous use. The present application describes multi-instrument access devices suitable for use in SPS procedures and other laparoscopic procedures.

### BRIEF DESCRIPTION OF THE DRAWINGS

FIGS. 1 through 14 illustrate a first embodiment of a multi-instrument access device, in which:

FIG. 1 is a perspective view a first embodiment of the multi-instrument access device, together with a clamp attachable to the multi-instrument access device for use in coupling the device to a supportive arm attached to an operating table or other operating room structure;

FIG. 2A is a partially exploded perspective view of a distal portion of the main tube;

FIG. 2B is a partially exploded perspective view of the proximal ends of the passive access tubes;

FIG. 3 is a partially exploded perspective view of the main tube and proximal fitting of the system of FIG. 1. The proximal fitting is shown in transverse cross-section;

FIG. 4 is a longitudinal cross-sectional perspective view of the main tube and proximal fitting;

FIG. 5 is a perspective view of the proximal fitting;

FIG. 6A is a perspective view of the instrument delivery tubes and actuators;

FIG. 6B is a plan view of the instrument delivery tube shown in FIG. 6A;

FIG. 6C is a plan view similar to FIG. 6B showing an alternate instrument delivery tube;

FIG. 7A is a longitudinal cross-section view of one of the members of the proximal fitting, showing the coupling member engaged in a first longitudinal position;

FIG. 7B is similar to FIG. 7A and shows the coupling member in a second longitudinal position;

FIGS. 8A-8C are elevation views of the proximal end of the proximal fitting and show the coupling members engaged in different ones of the longitudinal slots;

FIG. 9A is a perspective view similar to FIG. 1 but showing the instrument delivery tubes in a closed axial position;

FIG. 9B is a perspective view similar to FIG. 9A but showing the instrument delivery tubes in an intermediate axial position;

FIG. 10A is similar to FIG. 9A but shows the system using the alternate instrument delivery tubes shown in FIG. 6C in the closed axial position;

FIG. 10B is a plan view of the instrument delivery tubes of the embodiment of FIG. 10A;

FIG. 10C is similar to FIG. 10B but shows the instrument delivery tubes in the intermediate axial position;

FIG. 10D is similar to FIG. 10B but shows the instrument delivery tubes in the fully deployed position;

FIG. 11A is a longitudinal cross-section view of a proximal portion of an instrument delivery tube, an actuator, and a distal portion of a control tube;

FIG. 11B is an exploded view of the actuator of FIG. 11A;

FIG. 12A is a perspective view showing instruments in use in the multi-access system;

FIG. 12B is similar to FIG. 12A and shows deflection of an instrument used in an instrument delivery tube;

FIG. 13 is a perspective view of a proximal portion of an instrument delivery tube, an alternative actuator, and a distal portion of a control tube;

FIG. 14 is a perspective view of the alternative actuator of FIG. 13.

FIGS. 15-21 show a second embodiment of a multi-instrument access system in which:

FIG. 15 is a perspective view of the multi-instrument access device, showing the instrument delivery tubes in the closed position;

FIG. 16 is similar to FIG. 15 but shows the instrument delivery tubes in an expanded or deployed position;

FIG. 17 schematically illustrates positioning of the base through an incision in an abdominal wall;

FIG. 18 is a perspective view of the base;

FIG. 19 is a perspective view of the seal and associated features, without the instrument delivery tubes;

FIG. 20 is an exploded view of the seal and associated features of FIG. 19;

FIG. 21 is a perspective view showing an instrument delivery tube and actuator;

FIGS. 22 through 29 are figures showing a third embodiment of a multi-instrument access system in which:

FIG. 22 is a perspective view showing the multi-instrument access system in the deployed position;

FIG. 23 is a perspective view of the upper housing, base and detachable ports of the system of FIG. 10;

FIG. 24 is a partially exploded view of the components of FIG. 23;

FIG. 25 is an exploded view of the ports and plate;

FIG. 26 is a perspective view of the upper housing of the third embodiment, and may also be used in a modified version of the second embodiment;

FIG. 27 is a cross-section view of the upper housing;

FIG. 28 is a close-up view of a portion of the third embodiment, with the detachable ports removed to allow the bushings to be seen;

FIG. 29 is a perspective view of a bushing.

FIGS. 30-32B are figures illustrating a third embodiment, in which:

FIG. 30 is a perspective view of the proximal housing and instrument delivery tubes;

FIG. 31A is a perspective view of the proximal housing;

FIG. 31B is a cross-section view taken along the plane designated 31B-31B in FIG. 31A;

FIG. 32A is a perspective view of a portion of the instrument delivery tube, a guide, and a portion of the corresponding post;

FIG. 32B is similar to FIG. 32A but shows the instrument delivery tube axially rotated from the position shown in FIG. 32A;

FIG. 32C is similar to FIG. 32A but shows the instrument delivery tube advanced longitudinally from the position shown in FIG. 32A.

### DETAILED DESCRIPTION

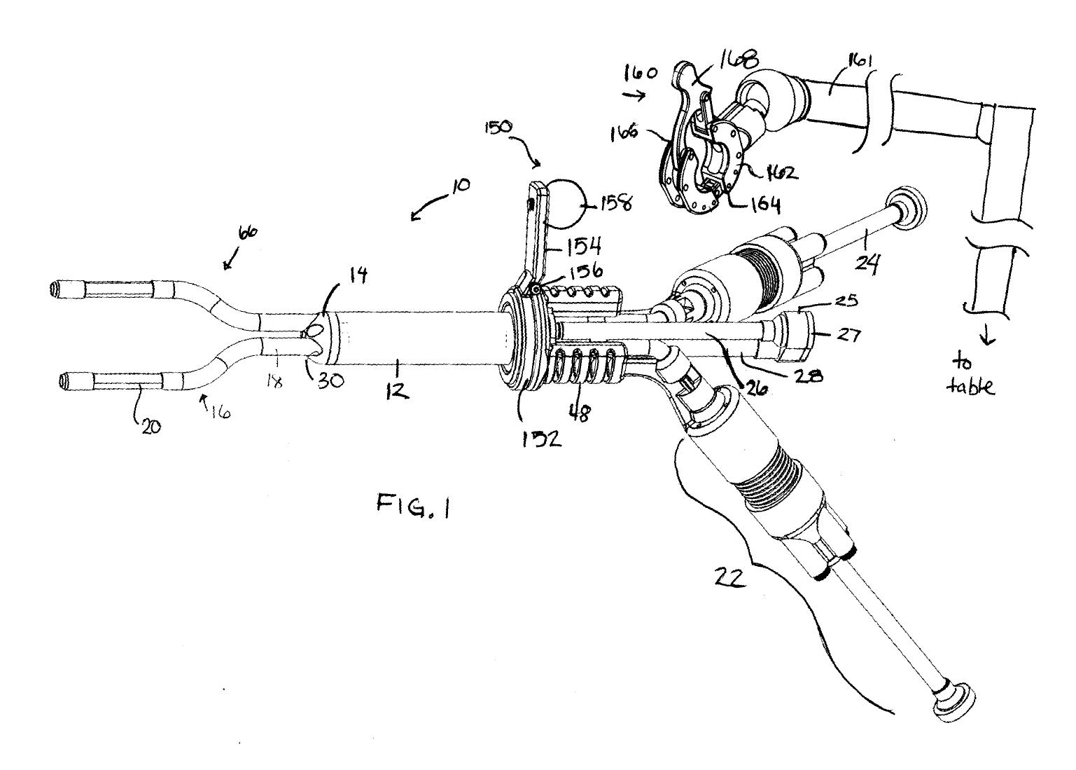

The accompanying figures illustrate multi-instrument access devices. In a first embodiment shown in FIG. 1, the access device 10 includes a base or main tube 12 positionable within an opening (e.g. an incision or puncture) formed in a body wall, namely through the skin and underlying tissue, to give access to a body cavity such as the peritoneal cavity. In some procedures, the opening may be formed through the umbilicus for purposes of cosmesis. During use, the tube remains disposed through the body wall opening and serves as the conduit through which the distal ends of multiple instruments are passed for use within the body cavity. In the illustrated embodiment, the main tube 12 provides access for introduction of up to four instruments into the body cavity via a pair of deflectable instrument delivery tubes 16, and a pair of passive access tubes 26, 28. Modifications to these embodiments within the scope of the invention can provide access for fewer or more than four instruments.

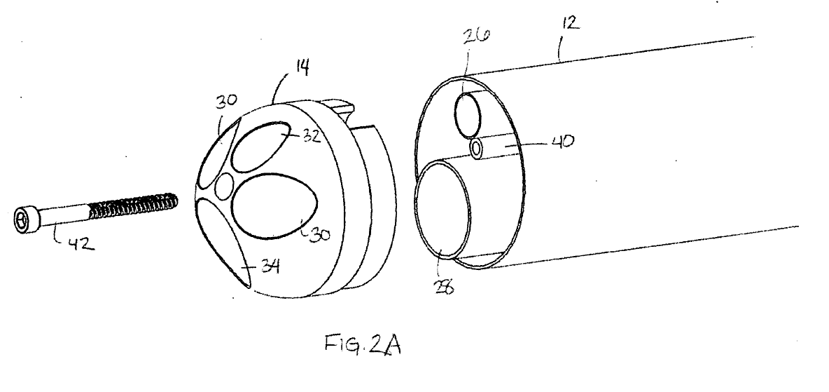

Main tube 12 is a rigid tube preferably having a single lumen. The outer diameter of the tube is preferably between 14-25 mm. The passive access tubes 26, 28 have proximal ends positioned external to the proximal end of the main tube 12 and distal ends disposed within the main tube 12 as shown in FIG. 2A. The portions of the access tubes 26, 28 extending through the main tube 12 may be integral with the proximal portions visible in FIG. 1, or each of the access tubes 26, 28 may be formed of one or more separate tubes longitudinally connected or coupled to one another. As shown in FIG. 2B, cross-slit seals 25 seal the lumen of the access tubes 26, 28, and septum type lead seals 27 (shown exploded from the access tubes) are positioned to seal against the shafts of instruments positioned within the tubes 26, 28. In the illustrated embodiment, the cross-slit seals 25 are part of a first cap that attaches to the seals, and the septum seals 27 are part of a second cap disposed on the first cap.

Referring again to FIG. 1, the distal end of the main tube 12 may include a partitioning element 14 that assists in maintaining the relative transverse positions of the instrument delivery tubes 16 and the shafts of instruments passing through the passive access tubes 26, 28. FIG. 2A shows the partitioning element 14 exploded from the main tube 12. In this embodiment, the partitioning element 14 defines first exit ports 30 through which the instrument delivery tubes 16 extend as shown in FIG. 1, and second and third exit ports 32, 34 longitudinally aligned with the passive access tubes 26, 28. A standoff 40 also extends through the main tube 12 and is coupled to the partitioning element 14 using a fastener 42.

In this embodiment, the partitioning element also forms an atraumatic distal tip for the main tube 12 due to the convex curvature of its outer surface.

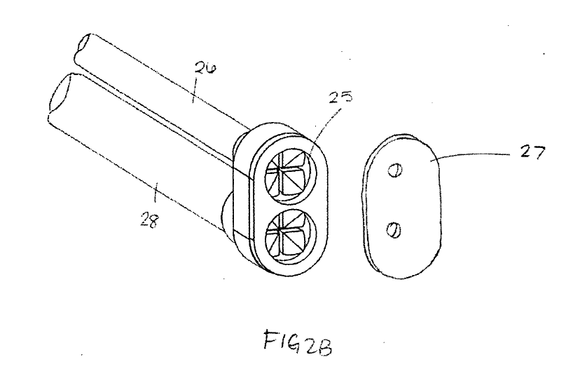

Referring to FIG. 3, a proximal seal 44 partially or fully disposed within the proximal portion of the main tube 12. The instrument delivery tubes 16 (not shown) and the passive access tubes 26, 28 (shown in cross-section) extend through corresponding openings in the proximal seal 44. O-rings 45 may be positioned at the openings in the proximal seal 44 to seal around the shafts of the instrument delivery tubes 16 and/or the passive access tubes 26, 28.

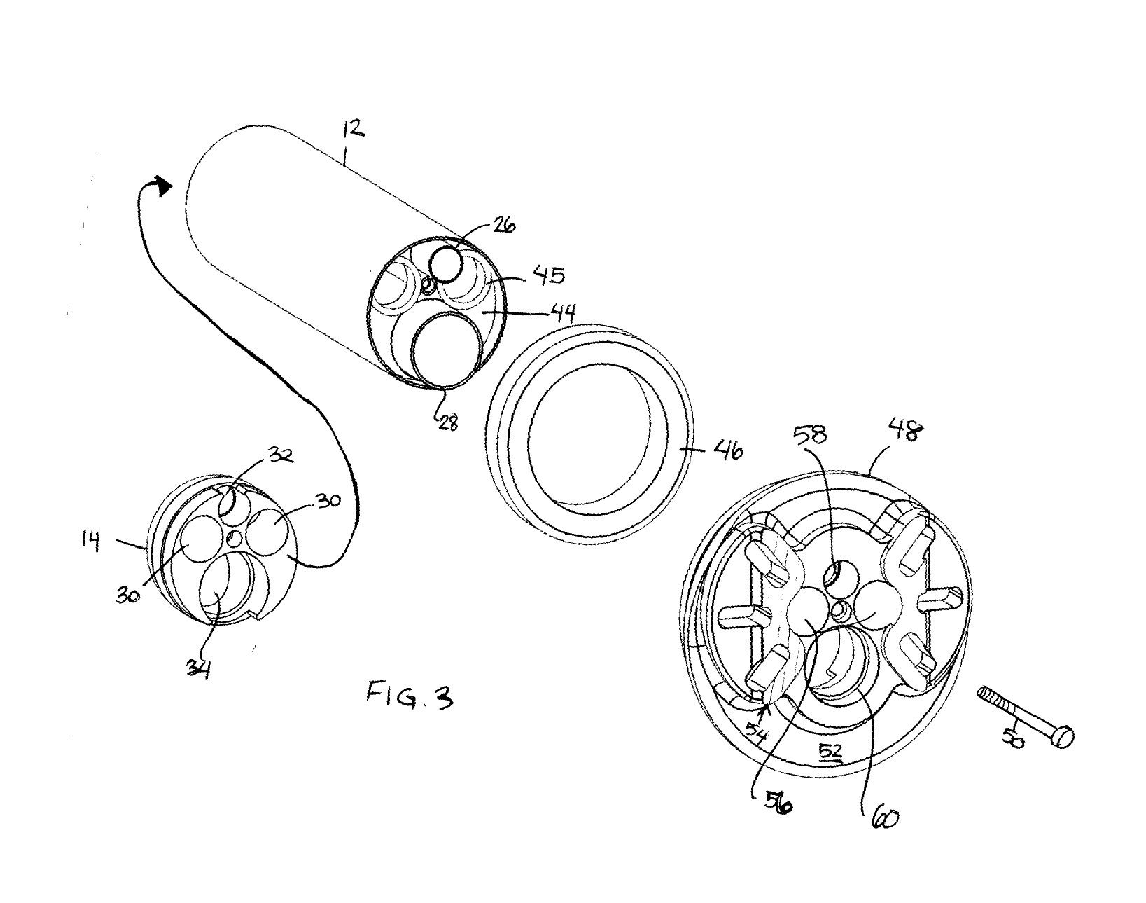

As shown in the longitudinal cross-section of FIG. 4, the proximal end of the main tube 12 extends into a proximal fitting 48. An annular seal 46 also disposed within the proximal fitting 48 forms a seal between the outer surface of the main tube 12 and the surrounding wall of the proximal fitting 48. A threaded fastener 50 (FIG. 3) extends through an opening in the proximal fitting 48 and is engaged with the bore of the standoff 40 so as to retain the proximal fitting 48 against the proximal end of the main tube 12.

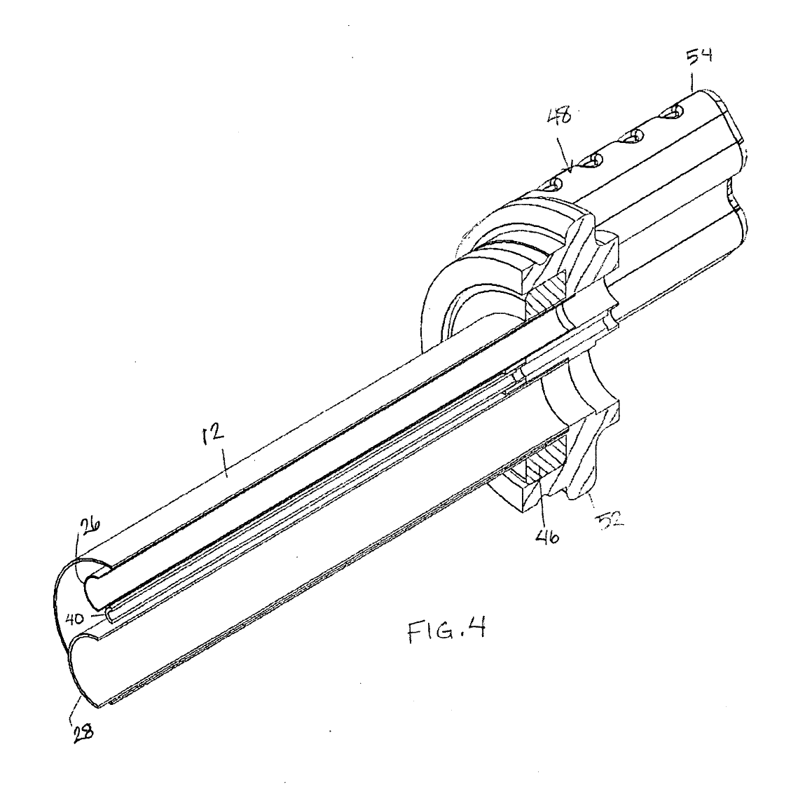

The proximal fitting includes a base 52 (FIG. 5) through which the instrument delivery tubes 16 and the passive access tubes 26, 28 extend. The base includes first openings 56 which accommodate the instrument delivery tubes 16 (not shown), and second and third openings 58, 60 which accommodate the inner tubes 26, 28. Members 54 extend proximally from the base 52 on opposite sides of the openings 56, 58, 60. FIG. 5 illustrates that each member 54 includes a plurality of longitudinally extending channels 62a, 62b, 62c each having an opening at the proximal face of the member 54. Circumferential slots 64a, 64b, 64c, 64d are formed in each member such that each longitudinal channel 62a-c intersects with each circumferential slot 64a-d.

Referring again to FIG. 1, the instrument delivery tubes 16 extend through the proximal fitting 48 and the main tube 12. In the illustrated embodiment, two such instrument delivery tubes are used, although alternative embodiments might use only one instrument delivery tube, while other embodiments might use three or more. Each instrument delivery tube 16 has a pre-shaped fixed curve or angle in its distal region 66.

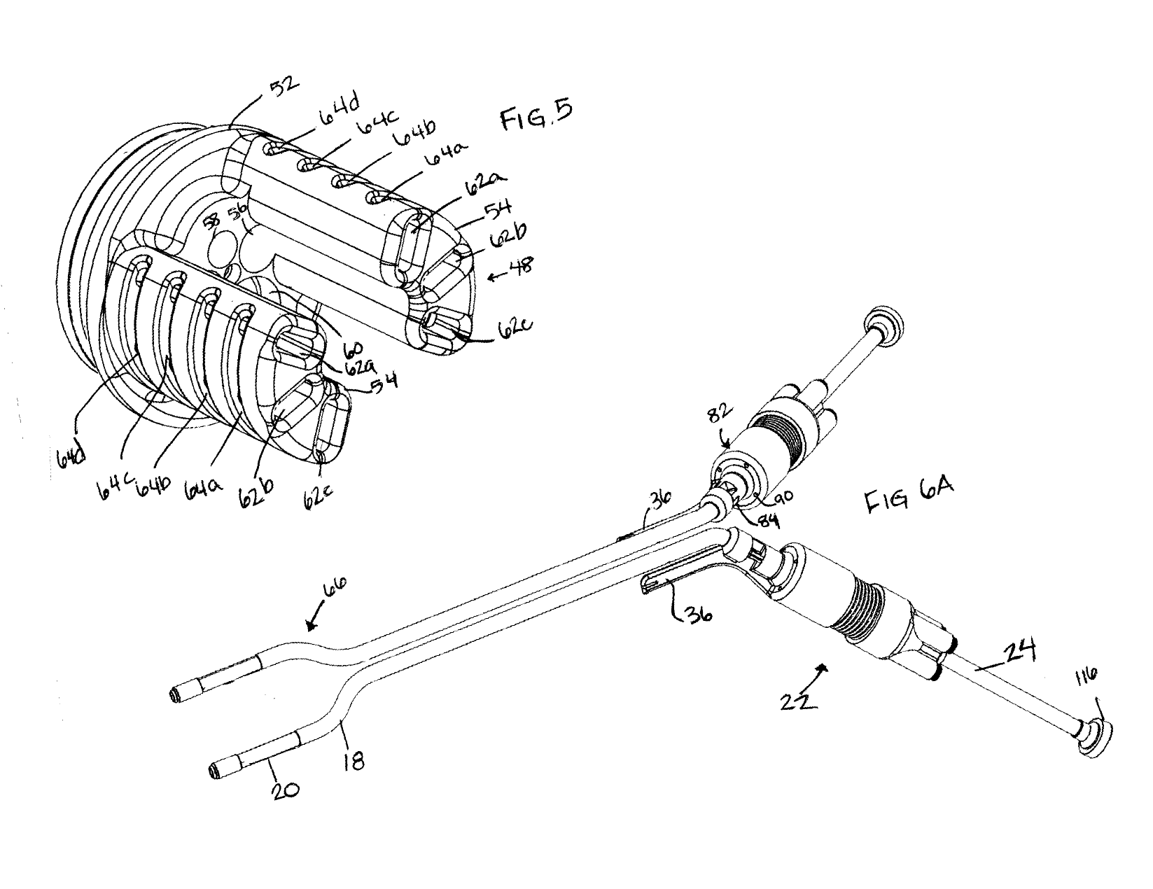

Referring to FIG. 6A, each instrument delivery tube 16 includes a rigid section 18 and a flexible section 20 extending from the distal end of the rigid section 18. Actuators 22 on the proximal portion of the access device 10 control deflection of the flexible distal sections 20 of the instrument delivery tubes 16 to allow manipulation of the operative ends of the instruments disposed within the instrument delivery tubes 16. As will be described in detail below, the distal ends of instruments to be deployed into the body cavity via the instrument delivery tubes are inserted into control tubes 24 on the actuators 22 and then advanced into and through the instrument delivery tubes. Manipulating the proximal handles of the instruments in turn moves the control tubes 24, causing corresponding deflection of the distal ends of the instruments.

Features of the instrument delivery tubes will next be described with respect to FIGS. 6A and 6B. Each instrument tube 16 includes a rigid tube 18 which may be formed of stainless steel or other rigid tubing. Each rigid tube 18 may be a singular tube, or a series of tubes coupled together. The stiffener tubes may all have the same size and/or geometry, or two or more different sizes and/or geometries may be used.

As shown in FIG. 6B, each rigid tube 18 is manufactured to have a fixed, preformed shape that includes a generally straight main section 70 and a distal region 66 which includes a bend to create a curved or angled section 68. The curvature of the bend in the curved or angled section may be continuous or compound, and it can be formed to occupy a single plane or multiple planes. The shape of the rigid tubes 18 separates the distal regions 66 of the instrument delivery tubes, allowing instruments passed through the instrument delivery tubes 16 to be used at common treatment site when the instrument delivery tubes 16 are in the deployed position.

The curved section 68 shown in FIG. 6B has an elongated S-shape, with a more proximal section that curves downwardly relative to the longitudinal axis of the main section 70 and a more distal section that curves slightly upwardly. It should be noted that the terms “downwardly”, “upwardly” etc are used with reference to the drawings and not with reference to particular structures inside or outside the body cavity. The distal region 66 may additionally have a second straight section 72 distal to the curved or angled section 68. In the FIG. 6A embodiment, the longitudinal axis of the straight section 72 is shown parallel to that of the straight main section 70, however it may alternatively diverge towards or away from the longitudinal axis of the base 12.

For the instrument delivery tube shown in FIG. 6B, the longitudinal axes of the straight shaft 70, curve 68 and distal end section 72 lie within a single plane, while a proximal bend section 74 of the tube 18 curves laterally out of that plane as well as downwardly. The proximal curvature of the proximal bend section 74 angles the actuators 22 away from one another in order to prevent interference between the handles of instruments used in the instrument delivery tubes 16 and instruments used in the passive tubes 26, 28.

Various alternative shapes for the tube 18 other than those shown in the illustrated embodiments may instead be used. For example, as shown in FIG. 6C, the bend may form a section 68a having a single curve or an angle extending from the straight shaft 70, rather than an s-shaped curve.

The instrument delivery tubes 16 also include flexible inner tubes 20 extending through the rigid tubes 18. Each inner tube 20 has distal and proximal sections 76, 78 extending beyond the distal and proximal ends, respectively, of the corresponding rigid tube 18. The inner tubes 20 can be made with or without a pre-formed curve or angle.

Each inner tube 20 includes a lumen for receiving an instrument that is to be used within the body. A plurality of actuation elements such as pull wires or cables 72 extend through pullwire lumens in the wall of the inner tube 20 and are anchored near its distal end in the distal section 76. In the preferred embodiment, each instrument delivery tube has four such wires arranged at 90 degree intervals. Other embodiments can utilize different numbers of pullwires, such as three pullwires equally spaced around each inner tube 20.

As will be discussed in detail below, the pullwires for each of the flexible tubes 20 are coupled to a corresponding one of the actuators 22 (FIG. 1), which act on the pull-wires to deflect the distal sections 76 of the flexible tubes 20. The inner tubes 20 are therefore constructed to be sufficiently flexible to allow the required deflection for instrument manipulation, while preferably also being resistant to kinking. In one embodiment, each flexible tube 20 is a composite tube formed using a PFTE inner liner lining the lumen, a thermal plastic sheath (having the pull wire lumens formed through it) overlaying the liner, a reinforcing layer over the thermal plastic sheath, and a second thermal plastic sheath over the reinforcing layer. In an alternate embodiment, the second thermal plastic sheath is eliminated and the reinforcing layer serves as the outer layer of the sheath. In yet another embodiment, the reinforcing layer may comprise the most inner layer of the tube. Various other embodiments, including those provided without reinforcing layers, or those having additional layers of reinforcing material or other materials can also be used.

Each such delivery tube 16 is longitudinally slidable and selectively retainable in a plurality of predetermined longitudinal positions to lengthen or shorten the amount of the instrument delivery tube extending from the main tube 12 into the body cavity. The instrument delivery tubes are also axially rotatable and selectively retainable in a plurality of predetermined axial orientations, allowing the user to choose the appropriate axial position of the curved distal region 66.

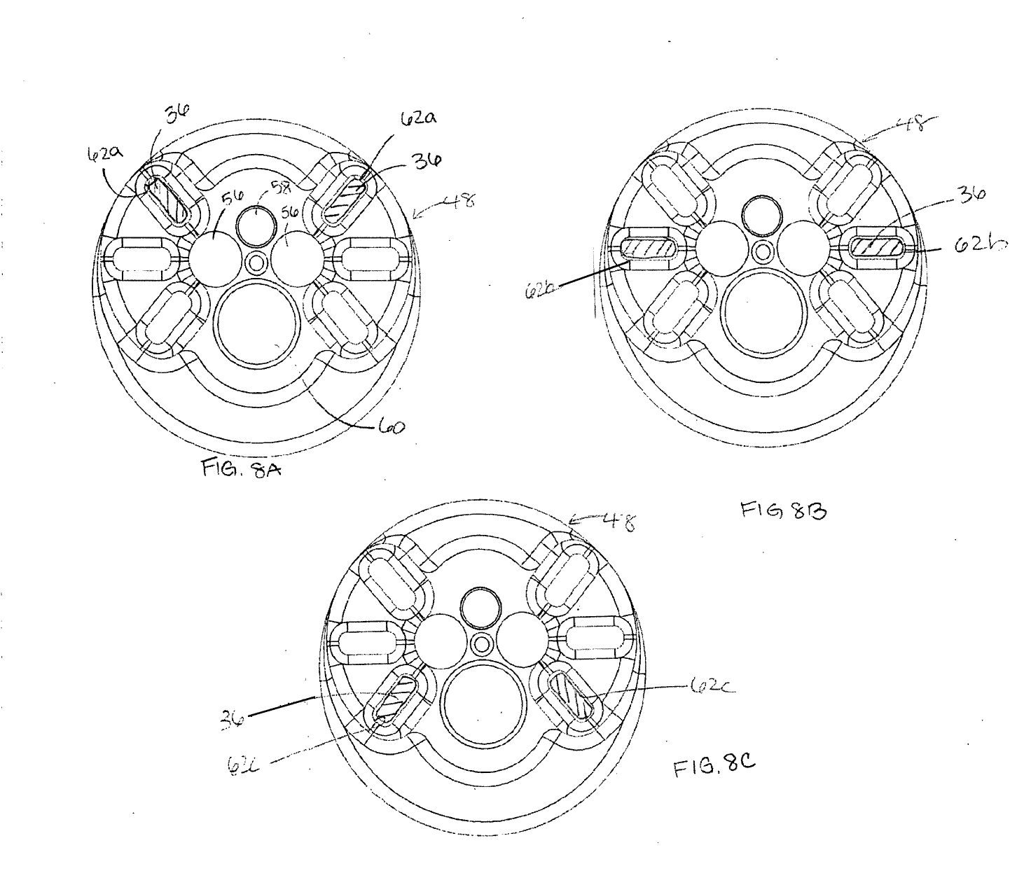

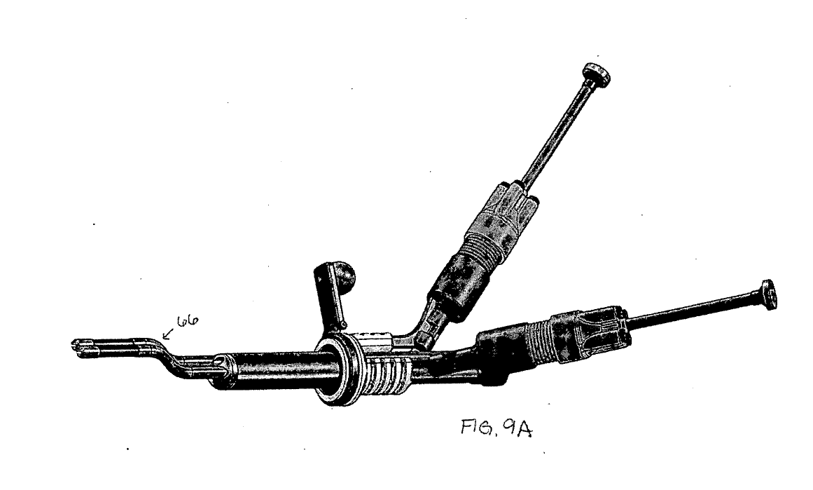

With regard to axial orientation, the instrument delivery tubes 16 can be retained in at least two pre-determined axial positions: (a) a closed or insertion position (FIGS. 9A, 10A and 10B) and (b) a fully open or deployed position (FIGS. 1 and 10D). The illustrated embodiment additionally includes the intermediate position shown in FIGS. 9B and 10C as a third pre-determined axial position at which the instrument delivery tubes can be retained.

In a preferred insertion position, the curved or angled distal regions 66 have a position that minimizes the maximum lateral distance between them. Thus, in FIG. 9A, the distal regions 66 are side by side and the curves of the distal regions 66 curve in parallel to one another. A similar arrangement is seen with the alternative instrument delivery tube shape shown in FIG. 10A. In the fully open or deployed position shown in FIGS. 1 and 10D, the curved or angled distal regions 66 are widely spaced apart. In this position, the lateral distance between the rigid sections of the instrument tubes in a direction orthogonal to the longitudinal axis of the main tube is at its maximum, and may be longer than the diameter of the main tube 12. In this position, the distal regions 66 of the two instrument delivery tubes 16 may share a common plane. For example, when viewed along the longitudinal axis of the main tube 12, the curved distal regions 66 may extend to 3 o'clock and 9 o'clock positions.

The third axial position (FIG. 9C) is an intermediate position in which the curved or angled distal regions are separated by an amount less than in the fully deployed position. In this position, the curved distal regions 66 of the two instrument delivery tubes 16, when viewed along the longitudinal axis of the main tube 12, may extend in the 2 and 9 o'clock positions, or in the 1 and 11 o'clock positions, for example. Although the illustrated system has three predetermined axial positions for each instrument delivery tubes, alternative systems may have only two predetermined axial positions, or they may have four or more such positions.

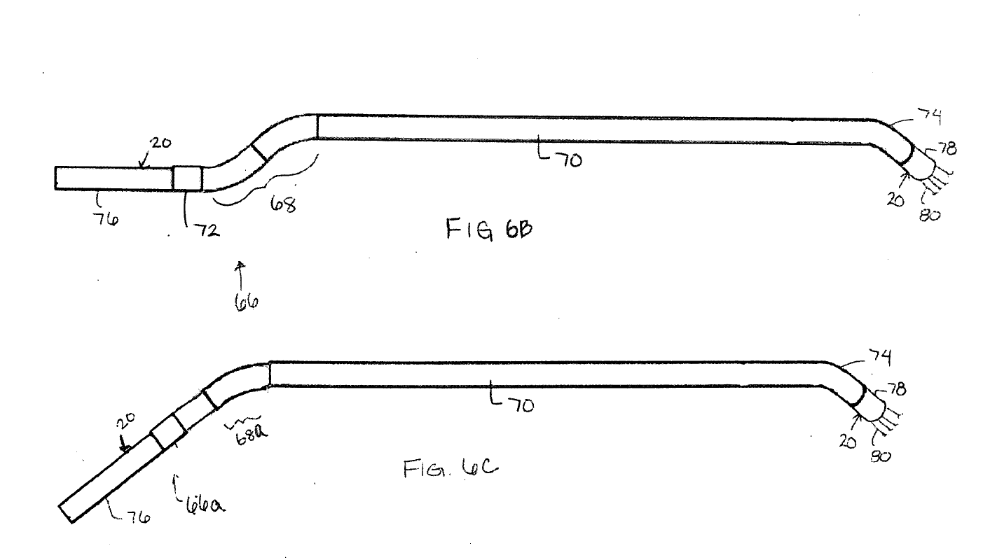

The system includes features allowing the user to retain the position of the instrument delivery tube at the selected axial or longitudinal position. In some embodiments, each instrument delivery tube 16 and/or its associated actuator 22 includes a member positionable in engagement with the proximal fitting 48 in order to fix the position of the instrument delivery tube 16 relative to the main tube 12. In the illustrated embodiment, this member takes the form of a coupling member 36 (FIG. 6A) insertable into a select one of the longitudinal channels 62a-c (FIG. 5) of the proximal fitting. Referring to FIG. 7A, a catch 38 is positioned at the distal end of the coupling member 36. The catch 38 extends laterally from a longitudinally extending spring element 39. The spring element 39 outwardly biases the catch 38 towards the adjacent circumferential grooves 64a-d. In the illustrated embodiment, the spring element 39 is defined by a longitudinal slot 41 in the coupling member 36.

When the catch 38 is disposed within a circumferential groove of a corresponding channel, such as groove 64c of channel 62c as in FIG. 7A, the spring bias of the catch 38 biases the catch into the groove and thus temporarily fixes the longitudinal position of the instrument delivery tube relative to the main tube 12. When the member 36 is advanced or retracted within the channel, the spring element 39 is caused to deflect as shown in FIG. 7B in response to contact between the catch 38 and the material between the circumferential grooves 64c, 64b, thus allowing the catch 38 to disengage from the groove 64c. Positioning the catch 38 in alignment with a selected one of the other grooves will cause the catch 38 to spring outwardly into engagement with the selected groove, again temporarily fixing the instrument delivery tube at a second longitudinal position.

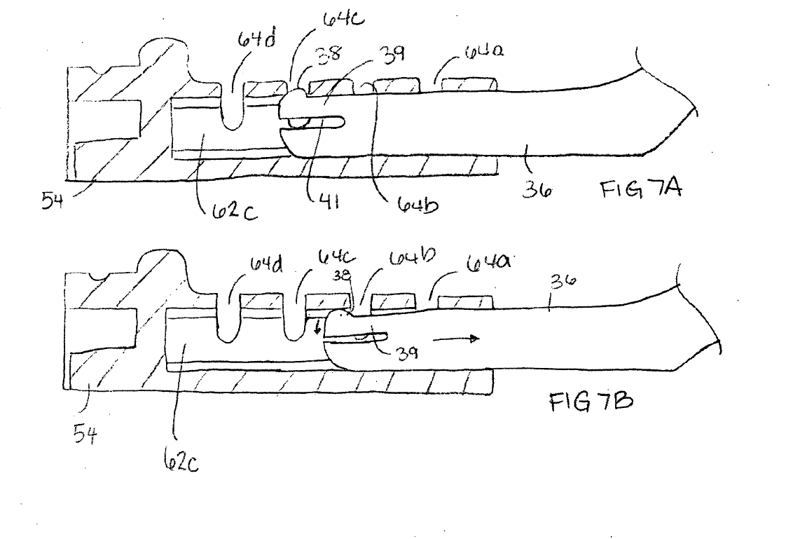

Each instrument delivery tube 16 is disposed in the main tube 12 with a portion of its straight section within the main tube 12 and with its curved or angled region 66 position distally of the main tube 12. Before the system is introduced into a body cavity, the coupling member 36 is preferably coupled to the proximal fitting 48. More specifically, the coupling member 36 is inserted into whichever of the longitudinal channels 62a, 62b, 62c corresponds to the desired axial orientation for the instrument delivery tube. For most applications, the coupling elements 36 for both instrument delivery tubes will be inserted into longitudinal channels 62a, as shown in FIG. 8A, in preparation for insertion of the system into the body cavity. This arrangement positions the curved distal regions of the instrument delivery tubes as shown in FIG. 9A or 10A, thus placing their distal portions in a streamlined arrangement for easy insertion into the body.

The user may also pre-select a longitudinal position for the instrument delivery tube 16 by advancing the catch 38 into engagement with a select one of the circumferential channels 64a-64d as discussed above with reference to FIGS. 7A and 7B. In doing so, the user is selecting how much of the distal end of the instrument delivery tube will extend from the main tube 12. Selecting the most proximal channel 64a will cause the shortest length of instrument delivery tube 16 to extend from the main tube 12, whereas selecting distal-most channel 64d will cause the longest length of instrument delivery tube 16 to extend from the main tube 12. If the user wishes to change the longitudinal position of an instrument delivery tube 16 during a procedure, s/he may do so by advancing or retracting it to the desired position and causing the catch 38 to engage the adjacent circumferential groove as discussed in connection with FIGS. 7A and 7B.

During the course of a procedure, the user may also choose to change the axial rotation of a given instrument delivery tube. For example, after the system has been inserted into to the body, the user may choose to rotate at least one of the instrument delivery tubes out of the position shown in FIG. 9A and into the position shown in FIG. 9B or FIG. 1.

To make this adjustment, the user extracts the coupling member 36 from a first one of the longitudinal channels 62a, 62b, 62c and re-inserts the coupling member 36 into a selected second one of the longitudinal channels corresponding to the desired axial position. Once the coupling member 36 is in the desired longitudinal channel, it is advanced until the catch 38 engages with the circumferential groove corresponding to the desired longitudinal placement of the instrument delivery tube 16. Inserting the coupling members 36 into channels 62b as shown in FIG. 8B will position the instrument delivery tubes in the positions illustrated in FIG. 9B or 10C. Inserting the coupling members 36 into channels 62c as shown in FIG. 8C will position the instrument delivery tubes in the positions shown in FIG. 1 or 10D. While these figures show the two instrument delivery tubes at the same axial and longitudinal positions, it is important to note that the instrument delivery tubes are independently adjustable both axially and longitudinally. Thus, each instrument delivery tube may be placed at a different axial and/or longitudinal position from that of the other instrument delivery.

In the illustrated embodiment, the longitudinal channels and circumferential slots enable the instrument delivery tubes 16 to be axially rotated between discrete axial positions and, once in a chosen axial orientation, to be longitudinally advanced/retracted between discrete longitudinal positions relative to the proximal fitting. Alternate embodiments might, however, be configured to allow axial rotation of an instrument delivery tube without altering the longitudinal position. Embodiments of this type will be described in connection with the third and fourth embodiments.

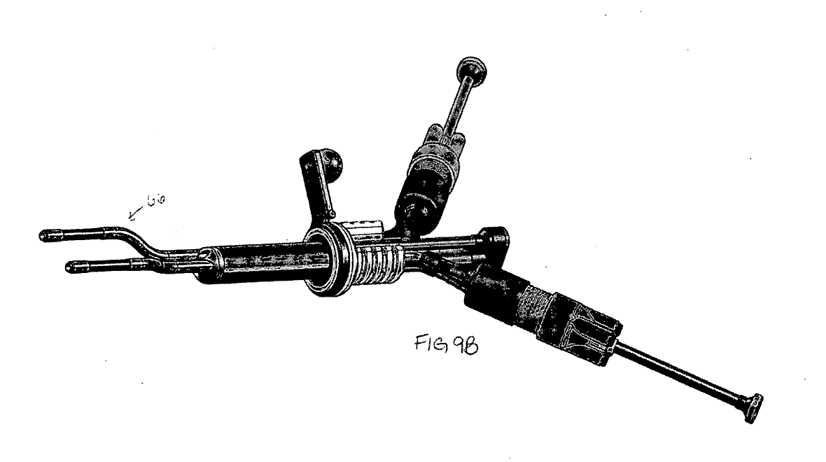

FIG. 11A shows a cross-section view of the proximal end of one of the instrument delivery tubes 16 and the corresponding actuator assembly 22. In general, the actuator assembly 22 includes a distal element 82, a proximal element 94, and a spring 96 extending between the distal and proximal elements. The rigid control tube 24 is coupled to the proximal element 94. The control tube 24 includes a lumen for receiving a medical instrument that is to be deployed through a corresponding instrument delivery tube 16. The control tube 24 may have a lubricious lining formed of PTFE or other suitable material so as to allow instruments inserted through the control tube to slide with ease.

Distal element 82 is mounted to the proximal end of the rigid tube 18 of the instrument delivery tube 16. The distal element includes a lumen 83. The proximal end of the rigid tube 18 is disposed in a fixed position within the lumen 83, with the proximal end 78 of the flexible inner tube 20 extending further proximally within the lumen 83. A plurality of openings or slots 84 (one visible in FIG. 11A) is formed in the distal element 82. Each slot 84 extends from the lumen 83 to the exterior of the distal element 82.

In a proximal portion of the distal element 82, the lumen 83 is surrounded by an inner cylindrical wall 86, which is itself surrounded by an outer cylindrical wall 88. The outer wall 88 defines a proximally facing cylindrical interior or receptacle, and also defines a cylindrical gap 92 between the two walls 86, 88. As best seen in FIG. 6A, a plurality of through holes 90 extend from the proximal end of the gap 92 (FIG. 11A) to the exterior of the proximal fitting 82. The through holes 90 and the slots 84 are radially aligned and correspond in number to the number of pullwires in the corresponding instrument delivery tube 16.

Referring again to FIG. 11A, proximal element 94 includes a wall 106 defining a distally-facing cylindrical interior or receptacle 108. A lumen 110 extends from the interior 108 to the proximal face of the proximal element 94. A plurality of pullwire lumen 112 extend through the proximal element 94, preferably in parallel to the lumen 110.

The spring 96 is coupled between the proximal element 94 and the distal element 82. In the illustrated embodiment, the distal end of the spring is disposed in the proximally-facing receptacle defined by outer wall 88 of the distal element 82, and the proximal end of the spring is disposed in the distally-facing receptacle 108 of the proximal element 94.

The spring 96 is a rigid spring formed of stainless steel or other suitable materials. Components extending through the spring define a sealed instrument passage between the proximal and distal elements 94, 82. A seal, such as the cross-slit seal 100 shown in FIG. 11A, is positioned in the lumen 83. This seal prevents loss of insufflation pressure through the actuator assembly 22 during times when there is not an instrument disposed in the corresponding instrument delivery tube. A length of flexible tubing, such as a Tygon tube 102, extends proximally from the seal 94. A connector 104 couples, and creates a seal between, the inner wall 86 and the tube 102.

The proximal end of the tube 102 extends into the lumen 110 of the proximal element 94. A tubular coupling 114 forms a sealed connection between the tube 102 and the control tube 24, which has a distal end disposed within the lumen 110. A seal 116 is positioned on the proximal end of the control tube 24. Seal 116 is preferably an elastomeric septum-type seal having an opening proportioned to seal against the shaft on an instrument positioned through the control tube 24.

The mechanism by which the actuator assemblies 22 control deflection of the flexible distal region of the corresponding instrument delivery tube will be next be described. As discussed in connection with FIG. 6B, pullwires 80 are anchored within the deflectable distal portion 76 of each flexible tube 20, and extend from the proximal portion 78 of the flexible tube 20 which, as noted in the discussion of FIG. 11B, is disposed within the distal element 82 of the actuator 22. The pullwires 80 then extend from the distal element 82 and are anchored to the proximal element 94. While other arrangements can be used, in the arrangement illustrated in FIG. 11, the pullwires 80 extend from the flexible tube 20, exit the distal element 82 via the slots 84, re-enter the distal element 82 via the throughholes 90, and extend through the spring 96 into the proximal element 94. The pullwires 80 are coupled to adjustment screws 118 on the proximal element 94. The adjustment screws are rotatable to adjust the sensitivity of the actuator by increasing or decreasing the tension on the pullwires.

Some prior art surgical access systems allow for pivotal motion of the shafts of instruments or instrument delivery cannulas relative to the longitudinal axis of the access port disposed within the incision, creating a fulcrum at some point along the shaft of the instrument. In preferred embodiments it is desirable to provide the access system with features that restrain the shafts of the instrument delivery tubes 16 against pivotable movement relative to the main tube 12, instead retaining the shafts of the instrument delivery tube such that the angular orientation of each instrument delivery tube remains fixed relative to the longitudinal axis of the main tube or base 12. With this arrangement, the straight proximal sections 70 of the instrument delivery tubes remain in parallel to one another and the curved section 68 of the rigid tubes are prevented from pivoting within the body. Thus, movement at the distal regions 66 of the instrument delivery tubes is limited to deflection of the flexible tube 20, axial rotation as described with reference to FIGS. 8A-8C, and longitudinal movement as described with reference to FIGS. 7A and 7B.

In the first embodiment, restraint against pivotable movement of the instrument delivery tubes 16 is provided by the connection between the proximal fitting 48 and the coupling members 36, and/or by the elongate bores 56 in the base 52 of the proximal fitting, and/or by the walls of the main tube 12 and/or the openings 30 in the partition 14.

To use the system, an incision is formed through the skin and underlying tissue. The distal end of the main tube 12 is inserted through the incision and into the body cavity. For the insertion step, the instrument delivery tubes 16 are preferably positioned as shown in FIGS. 9A and 10A for ease of insertion. The body cavity is inflated using a source of inflation gas as is common in laparoscopy. An insufflation port may be provided in one of the instrument delivery tubes or ports 26, 28 or elsewhere in the device to allow a source of gas to be coupled to the access device for use in inflating the body cavity. As discussed, seals are provided for each port 16, 26, 28 to seal the ports against loss of inflation pressure around the shafts of instruments positioned in the ports, as well as to minimize loss of inflation through ports not occupied by instruments at any given time.

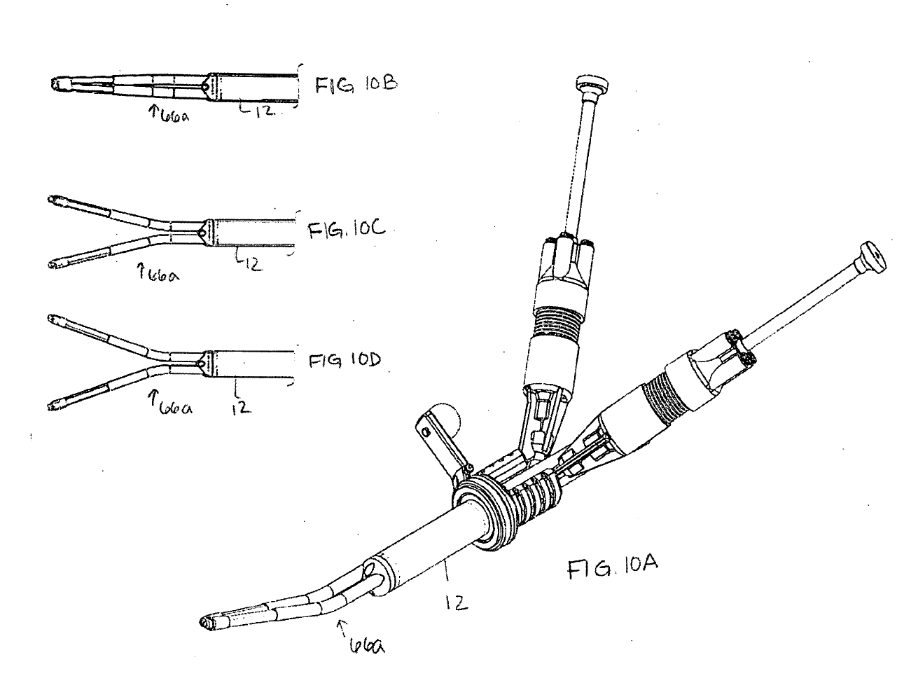

The surgeon will select instruments needed to perform a procedure within the body cavity. For example, referring to FIG. 12, a first instrument 120 is chosen through deployment and use through a first one of the instrument delivery tubes 16, and a second instrument (not shown) is selected for use through a second one of the instrument delivery tubes. A third instrument 122, which may be one with a rigid shaft, is positioned through the port 26, with its distal end passing into the body cavity through opening 32 in the partition 14. A fourth instrument 124 (e.g. a rigid endoscope) is advanced into the body cavity through port 28 and opening 34.

To deploy an instrument through an instrument delivery tube 16, the distal end of the instrument I is inserted into to the port 116 at the proximal end of the control tube 24. The instrument is advanced to pass the distal end through the actuator 22 and through the instrument delivery tube 16 until it extends from the distal end of the flexible tube 20. The instrument 120 may then be use for diagnosis or treatment at a treatment site in the body cavity.

When it becomes necessary for the surgeon to deflect or articulate the distal end of the instrument 120, s/he intuitively moves the handle of that instrument, causing the control tube 24 and thus the proximal element 94 to move with it. The instrument 120 may be provided with a rigid section 126 extending from the handle to optimize force transfer from the instrument 120 to the control tube 24. Movement of the control tube will cause the proximal element 94 of the actuator 22 to move relative to the distal element 82, causing the spring 96 to bend and tensioning the pullwires in accordance with the angle of the proximal element relative to the distal element. The pullwires deflect the distal portion 76 of the flexible tube 20 portion of the instrument delivery tube 16, causing corresponding deflection of the distal end of the shaft of the instrument disposed within the instrument delivery tube. Thus, to lower the distal end of the instrument as shown in FIG. 12B, the user will raise the instrument handle 120, moving the proximal portion 94 upwardly relative to the distal portion 82. This will thus apply tension to the lower pullwires, causing downward deflection of the instrument delivery tube as well as the distal end of the instrument. Lateral movement of the instrument shaft to the right will tension the corresponding side pullwire to cause the distal portion of the instrument delivery tube to bend to the left. The actuator system allows combinations of vertical and lateral deflection, giving 360° deflection to the instrument delivery tube. The user may additionally advance/retract the tool longitudinally within the instrument delivery tube, and/or axially rotate the instrument within the instrument delivery tube when required.

Instruments suitable for use with the instrument delivery tubes include those described in co-pending U.S. application Ser. No. ______, filed Jul. 28, 2009, (Attorney Docket No. TRX-2100), entitled Flexible Dissecting Forceps, and U.S. application Ser. No. ______, filed Jul. 28, 2009, (Attorney Docket No. TRX-2400), entitled Flexible Medical Instruments, each of which is incorporated herein by reference.

It should be noted that the deflectable instrument delivery tubes and actuators described in connection with FIGS. 10-12B may be used with any other type of access system suitable for use in giving access to a body cavity. For example, the instrument delivery tubes and actuators may be used in trocars or other laparoscopic ports or access devices now known or developed in the future. Moreover, the instrument delivery tubes may be provided with alternative actuation systems for the pullwire. Various pullwire actuation systems are known to those skilled in the art and may be adapted for use with the instrument delivery tubes 16.

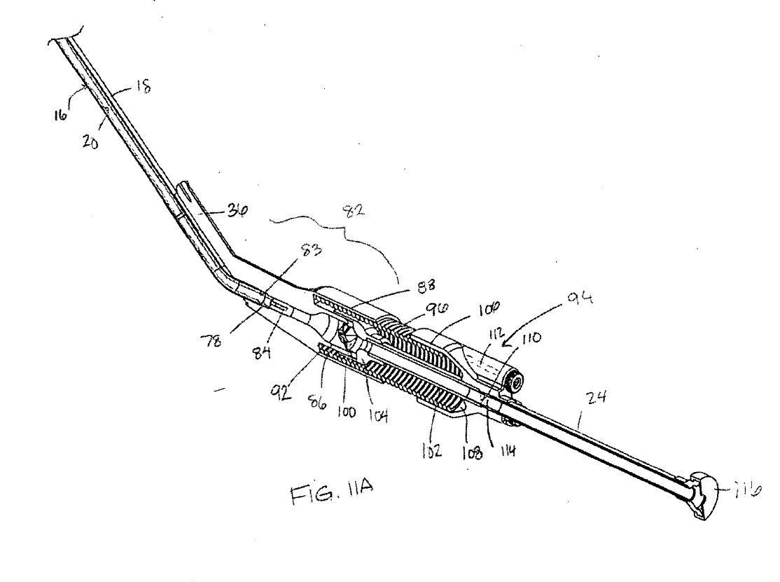

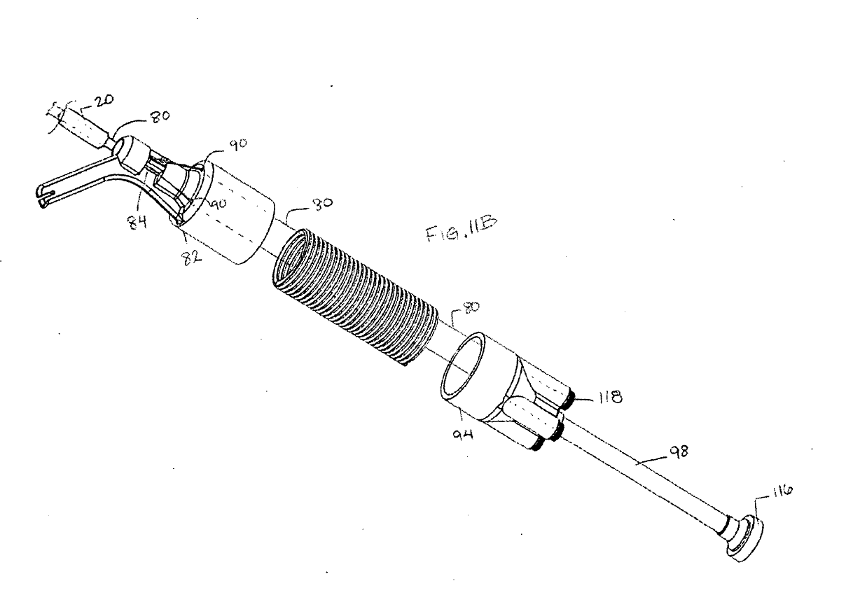

FIG. 13 shows the proximal portion of an instrument delivery tube 16 equipped with one type of alternative actuator 22a. In this embodiment, the features of the instrument delivery tube 16 are similar to those described earlier and thus will not be repeated. Details of the actuator 22a are most easily seen in the exploded view of FIG. 14. The actuator 22a includes a control tube 24a having proximal entry port/lead seal 116a for receiving a medical instrument that is to be deployed through the instrument delivery tube 16a. A proximal gimbal portion 128 is positioned distally of the control tube 24a and includes a proximal opening 130 which receives the distal end of the tube 24a. The proximal gimbal portion 128 also includes a distally facing socket 132. A distal gimbal portion 134 includes a proximally facing ball 136 disposed within the socket 132 and a tubular housing 138 extending distally from the ball 136. The ball 136 has a proximally-facing opening 142. A valve 144, which may be a cross-slit duck bill valve, is disposed within the tubular housing 138. The valve 144 functions to seal the actuator against loss of inflation pressure when no instruments are positioned through it.

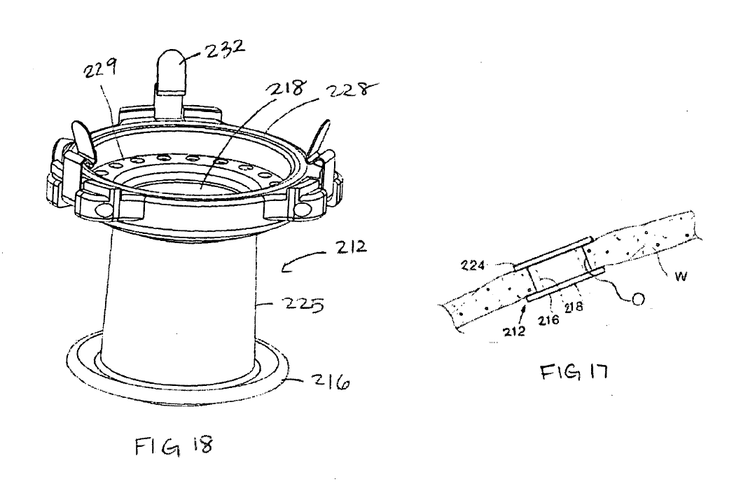

A fitting 146 (FIG. 13) connects the instrument delivery tube 16a to the proximal gimbal section 134. Pullwires 80 exiting the proximal end of the instrument delivery tube 16 exit the distal gimbal section 138 through slots 148 and into engagement with the proximal gimbal section 128. The pullwires are coupled to the proximal gimbal section 128 and secured using nuts 118 in a manner similar to that described with the first embodiment. In a slight modification to the FIG. 13 embodiment, nuts 118a are replaced by ball pivot mounts 118a as shown in FIG. 21 to create a universal joint for each pullwire. Each pullwire 80 is attached by a tensioning nut housing 119 to a ring 121 that encircles the corresponding ball pivot mount 118a and that has full freedom to move in any direction over the surface of the ball pivot.

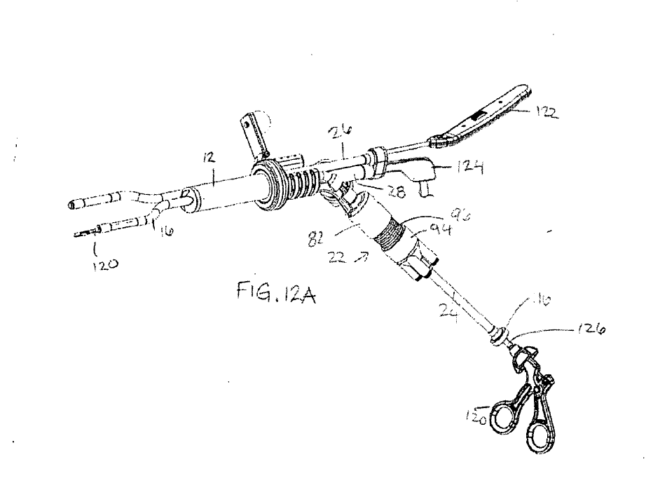

Referring again to FIG. 15, a Tygon tube (not shown) may extend through the actuator, coupled to the control tube 24a and the instrument delivery tube 16a in a manner similar to that described in connection with FIG. 10 to maintain a sealed lumen from the proximal end of the control tube 24a to the distal end of the instrument delivery tube 16a.

During use of the actuation system, the shaft of an instrument (e.g. instrument 120 shown in FIG. 12A is inserted through the control tube 24a (FIG. 13), proximal gimbal portion 132, distal gimbal 134 portion etc. and through the instrument delivery tube 16a such that its operative end exits into the body cavity. To deflect the distal end of the instrument, the user moves the handle of that instrument, causing the control tube 24a to move with it. The socket of proximal gimbal portion 128 will move over the ball surface of the distal gimbal portion 134, thus tensioning the pullwires in accordance with the angle of the proximal gimbal portion relative to the distal gimbal portion. The distal portion of the instrument will deflect accordingly as a result of the action of the gimbal on the pullwires of the instrument delivery tube.

Referring again to FIG. 1, the access system includes a mount 150 allowing the system to be engaged by a clamp on a supportive arm for supporting the system 10 without requiring the system 10 to be held in place by operating room personnel. In the illustrated embodiment, the mount 150 includes a collar 152 disposed on the proximal fitting 48 or tube 12 and an arm 154 extending from the collar 152. An adjustment screw 156 allows the grip of the collar on the tube 12 to be tightened or loosed. A spherical coupling 158 is disposed on the arm 154. The spherical coupling 158 is shaped to be received and engaged by a connector 160 provided on an arm 161 mounted to the operating table (not shown) or to another operating room fixture such as the ceiling or a cart.

The illustrated clamp 160 comprises a collar having semi-annular segments 162. Each segment 162 includes a first end 164 coupled to the other one of the segments, and a second end 166 hinged to a latch 168. The collar has an unlatched position shown in FIG. 1 in which the latch 168 is pivoted outwardly to separate the ends 166 of the semi-annular segments 162. The latch is inwardly pivotable to place the collar in a latched position, in which the ends 166 are drawn closer together and retained in the closed position by the latch 168.

To couple the spherical coupling 158 to the clamp 160, the clamp is placed in the unlatched position and disposed around the mount 158. The user places the system 10 in the desired three-dimensional orientation and then closes the latch 168 to capture the spherical mount 158 between the segments 162.

If the tube 12 needs to be rotated around its longitudinal axis during a procedure or preparation for a procedure in order to collectively adjust the positions of the instrument delivery tubes and passive tubes, the collar 152 of the mount 50 is loosened, the tube 12 is axially rotated, and the collar is retightened.

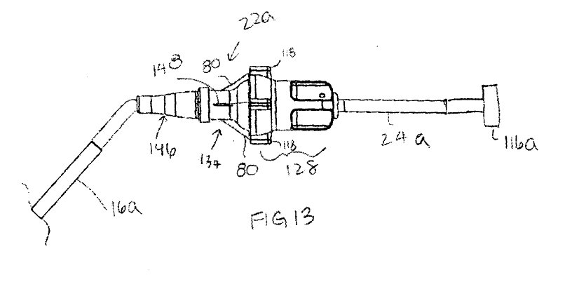

FIG. 15 shows a second embodiment of a multi-instrument access device 200. The access device 200 includes a base 212 positionable within an opening (e.g. an incision or puncture) formed in a body wall, through the umbilicus or elsewhere. An upper housing or seal 214 is attachable to the base 212 and positioned such that it is disposed outside the body wall during use. FIG. 17 schematically illustrates the base 212 in an incision in a body wall.

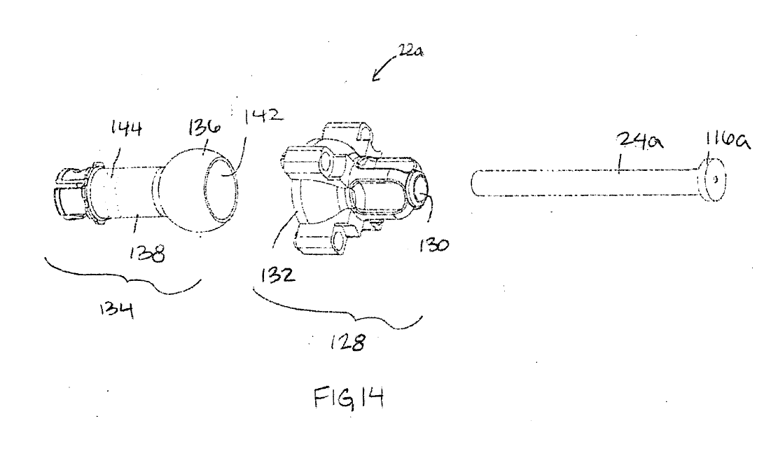

Referring to FIG. 18, base 212 is a generally hollow or tubular member having a wall 225 defining a lumen 218 and a distal flange 216 surrounding the distal opening of the lumen. The flange and distal opening may be circular, elliptical, or any other shape suitable for insertion into an opening in the body wall. The base 212 is preferably constructed of a flexible material that allows the base 212 to be pinched or flattened into a smaller profile for insertion through the opening in the body wall, and that will preferably restore the base to its original shape and size after compression is released.

Flange 216 has a width that will define a sufficient margin around the border of the opening in the abdominal wall to prevent its inadvertent withdrawal from the opening during use. Although flange 216 is shown as a fully circumferential member, alternate elements that are not fully circumferential (e.g. two or more flange segments), may alternatively be used to perform the same retention function. By including a broad flange, the base is able to retract peritoneal tissue away from the base port, keeping the tissue from obstructing access, preventing tools and/or implants from inadvertently slipping between the abdominal wall and the peritoneal tissue. The flange 216 may also form a seal around the incision to help maintain insufflation pressure within the abdominal cavity.

The base 212 and upper housing/seal 214 are preferably separate pieces attachable to each other during use. The seal 214 includes a first engaging portion which in this embodiment takes the form of a flange 226. The base 212 includes a second engaging portion positioned to engage the first engaging portion. In the illustrated embodiment, the second engaging portion includes a ring 228 on the base 212. The flange 226 of the seal 214 seats against and makes sealing contact with the ring 228. Clips 232 (preferably two or more) on the ring 228 are used to secure the base 212 to the seal 214.

The base 212 may be placed in the opening in the body wall before the seal 214 is coupled to the base. This is particularly beneficial where an initial step in the procedure may involve an instrument or implant that is too large for the ports 220. For example, where the access device 200 is to be used to implant a lap band or a Swiss lap band of the type used to induce weight loss, the lap band may be dropped through the lumen 218 in the base 212 and into the operative space. Then, once the seal 214 has been coupled to the base 212, the implant may be retrieved from within the operative space using an instrument passed through the seal 214. To position the flexible base 212 in the incision, it is folded or pinched and inserted into the opening O in the abdominal wall W and advanced until distal flange 216 is disposed beneath the abdominal wall W. The base 212 is allowed to unfold such that the wall surrounding the base contacts the edges of the opening O, keeping the opening open for access by instruments.

As shown in FIG. 17, a proximal flange 224 (or equivalent structure) is positioned to contact the skin surrounding the opening in the abdominal wall, to prevent the access device from inadvertently being pushed into the body cavity during use. This structure may be provided on the distal portion of the seal 214 or on the proximal portion of the base 212.

Referring again to FIG. 15, seal 214 includes a plurality of ports 220a, 220b extending proximally from the base 212. The ports 220a, b are tubular elements having proximal openings 222. The ports 220a, 220b are configured to receive instruments for use in performing a procedure within the body cavity. Valves (not shown in FIG. 15) are positioned within the ports 220a, 220b so as to maintain insufflation pressure within the abdominal cavity during use of the access device 200. These valves may include a duckbill valve for preventing loss of pressure when no instruments are disposed in the ports 220a, 220b as well as annular seals or septum seals for sealing against the shafts of instruments passed through the ports 220a, 220b. The ports 220a, 220b may be flexible to allow them to pivot relative to the base 212 when instruments deployed through them are being used in the body cavity.

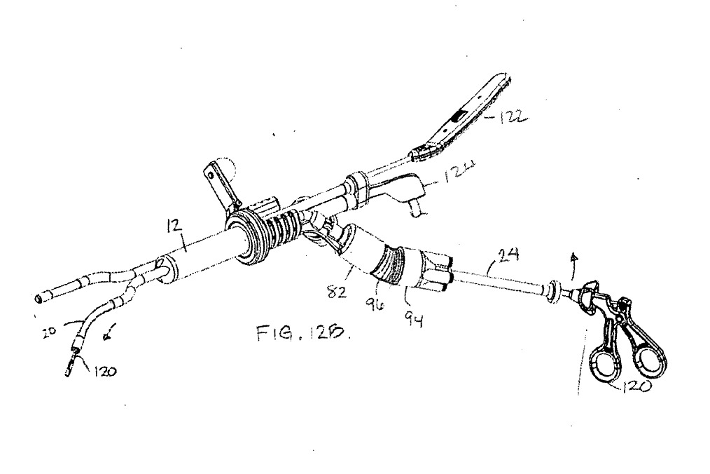

The other two ports 220c are provided to have instrument tubes 16b extending through them or coupled to them. The ports 220c may comprise passages through the upper housing, such as openings into the interior of the seal 214. Each instrument tube 16b extends through a port 220c and through the seal and base, and extends out the distal opening in the base. Each instrument tube 16b is provided with a pre-shaped curve in its distal region 252. The instrument tubes have a closed position shown in FIG. 15 in which the distal regions 252 are positioned to minimize the lateral distance between them. In the closed position, the distal regions 252 may cross as shown. The instruments tubes further have an open or deployed position shown in FIG. 16 in which the curved distal regions are oriented such that instruments passed through the lumens of the instrument tubes can access a target treatment site. In this position, the longest lateral distance between the instrument tubes may be longer than the diameter of the wall of the base.

In one configuration, each instrument tube 16b includes a rigid stiffener tube 254 having the pre-shaped curve. The rigid tubes may all have the same size and/or geometry, or two or more different sizes and/or geometries may be used. The curve in any given instrument tube may be continuous or compound, and it can be formed to occupy a single plane or multiple planes.

In one embodiment shown in FIG. 21, each rigid tube 254 has a generally straight main section 255a, and a pre-shaped curve 255b that generally curves outwardly from the main section 255a and that then (optionally) curves slightly inwardly. The curve(s) of the distal section may lie within the plane containing the main section 255a as shown, or the curve(s) may exit that plane. The curvature of the rigid stiffener tubes 254 serves to orient the distal sections 252 towards one another such that instruments passed through the instrument delivery tubes 16b can access a common treatment site when the instrument delivery tubes 16b are in the deployed position. The rigid stiffener tubes may be formed of stainless steel or other rigid tubing.

Flexible inner tubes 257 extend through the rigid stiffener tubes 254. Each inner tube 257 has a distal section 257a that extends distally from the corresponding rigid tube, and a proximal section 257b that extends proximally from the corresponding rigid tube. The inner tubes 212 can be made with or without a pre-formed shape.

Each inner tube 257 includes a lumen for receiving an instrument that is to be used within the body. Also provided on each inner tube is a plurality of pull wires 276 extending through pullwire lumens and anchored near the distal end of the inner tube 257. In the preferred embodiment, each instrument delivery tube has four wires such arranged at 90 degree intervals. Other embodiments can utilize different numbers of pullwires, such as three pullwires equally spaced around each inner tube 257.

The set of pullwires for each of the inner tubes 257 is coupled to a corresponding actuator 259, which may be manipulated to deflect the distal sections 257a of the flexible tubes 257 as discussed in connection with the first embodiment. The actuators 259 may be similar to the actuators described with reference to FIG. 11 or 14, or alternative actuators may be used. By deflecting the distal sections of the instrument delivery tubes 257, the flexible instruments extending through them are deflected within the body into desired positions and orientations.

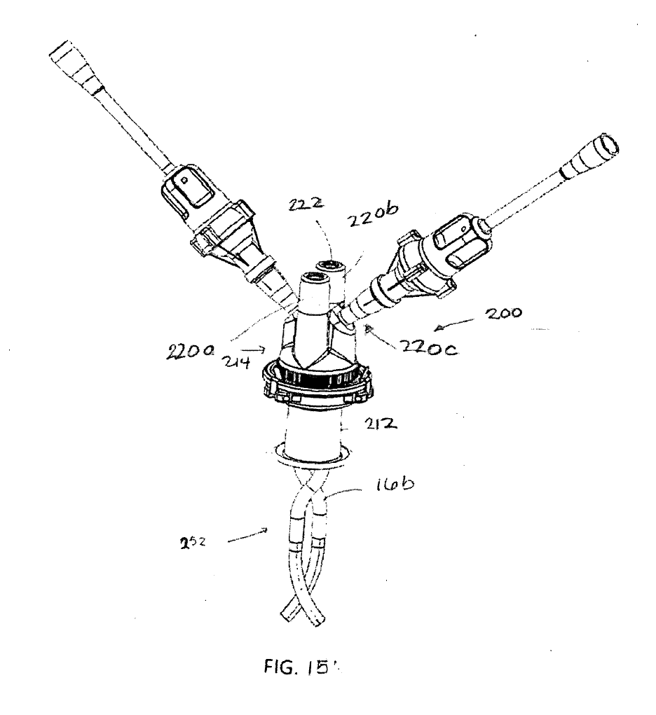

The rigid tubes of the instrument delivery tubes 16b are axially rotatable to a closed or insertion position, shown in FIG. 15, in which the instrument tubes have a more streamlined orientation for passage through the incision during insertion and withdrawal of the access system. Various mechanisms may be used for axially rotating the instrument tubes. In the embodiment illustrated in FIGS. 15-21, the rigid tubes 254 of the instrument delivery tubes are mounted at their proximal ends to gear members 278 or to bushings 277 attached to the gear members. The gear members 278 have teeth at their outer periphery. A rotatable collar 261 which has teeth along its inner periphery is positioned surrounding the gear members, such that teeth of the gear members 278 mesh with teeth of the rotatable collar 261. With this arrangement, rotation of the collar will cause simultaneous rotation of the rigid tubes 254 and thus the instrument delivery tubes 16b between the deployed and the insertion positions. The connection between the gear members or bushings and the rigid tube prevent pivotable movement of the rigid tubes relative to the base.

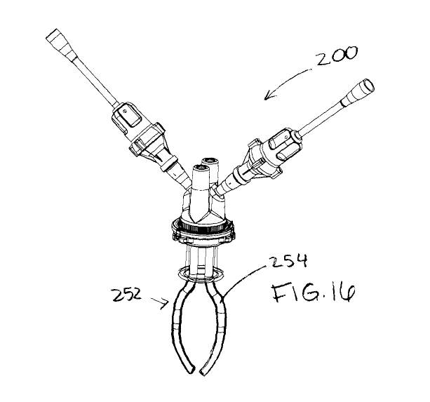

Referring to FIG. 20, the outer circumference of the collar 261 is exposed through a slot 279 in the upper housing 214 to permit a user to rotate the collar 261 relative to the upper housing 214. Support members connecting the portion of the upper housing disposed above the slot to the portion of the housing below the slot are not visible in the drawings. In an alternative embodiment, the collar 261 may be positioned between the upper housing 214 and the base 216. In either case, seals may be positioned above and below the collar to minimize loss of insufflation pressure between the collar and the upper housing and/or base.

A plate 280 may be positioned beneath the gear members and the collar 261 so as to support the collar. In one embodiment, the plate may be arranged to seat within the proximal end of the base 212, such as on the ledge 229 within the proximal opening of the base shown in FIG. 18. Alternatively, the plate may be mounted within the distal portion of the upper housing or seal 214. Holes 281 are arranged on the plate to receive the stiffener tubes 254, and holes 282 are similarly positioned to receive instruments inserted through the ports 220a, 220b. As with the first embodiment, the rigid tubes 254 of the instrument delivery tubes in the second embodiment are mounted to the system in a manner that prevents them from pivoting relative to the housing 212, 214. In this embodiment, restriction against pivoting is provided by the connection between the proximal ends of the rigid tubes and the gear members 278.

The second embodiment preferably includes a mount (not shown) such as the mount 150 of FIG. 1 allowing the system to be engaged by a clamp on a supportive arm attached to an operating table, ceiling mount, side cart, or other structure.

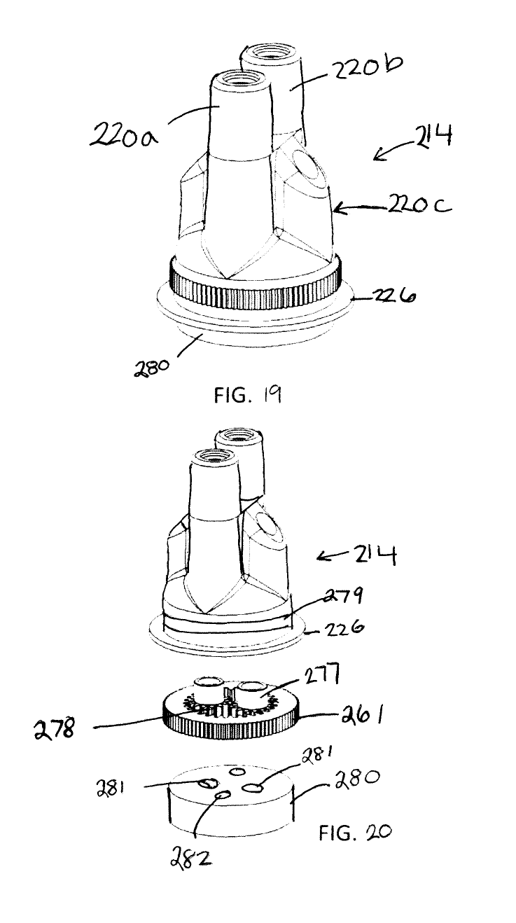

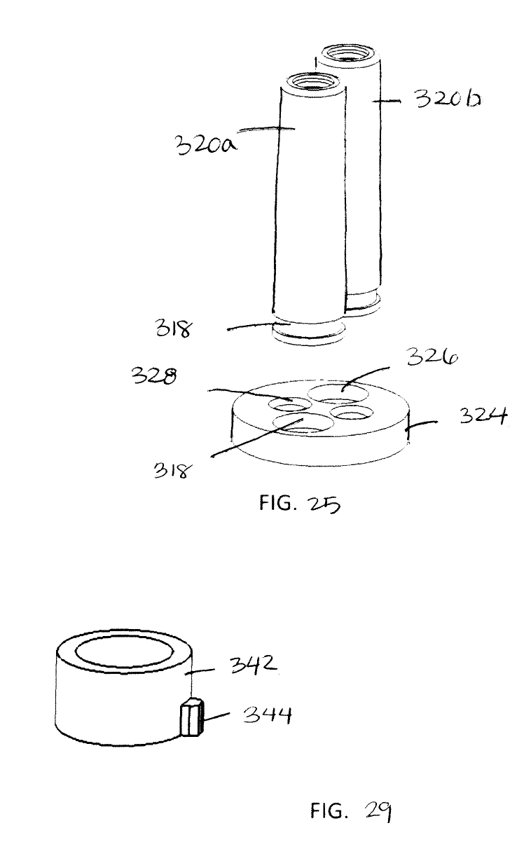

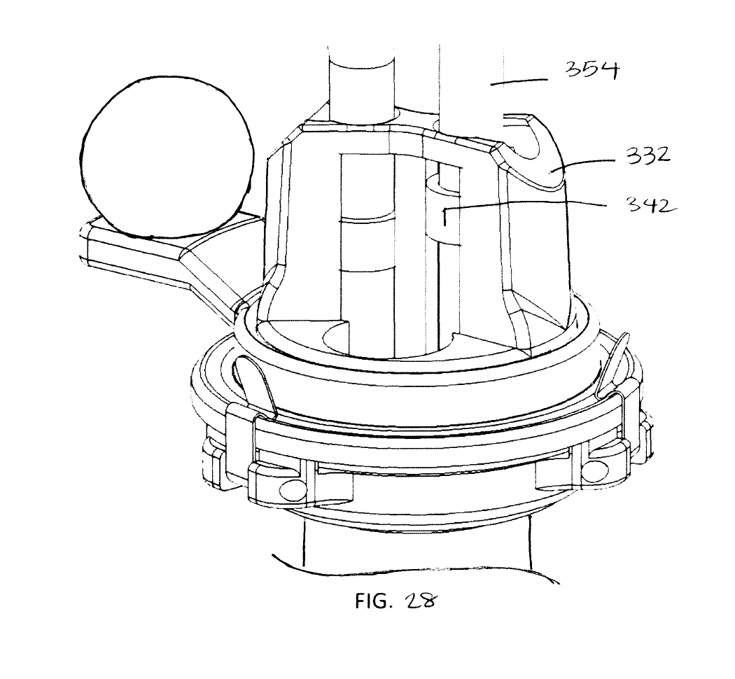

A third embodiment is shown in FIGS. 22 through 29 and has many features similar to those shown in the FIG. 15-21 embodiment. However the FIG. 22-29 embodiment, includes a different mechanism for axially rotating the instrument delivery tubes and it has an alternative upper housing configuration.

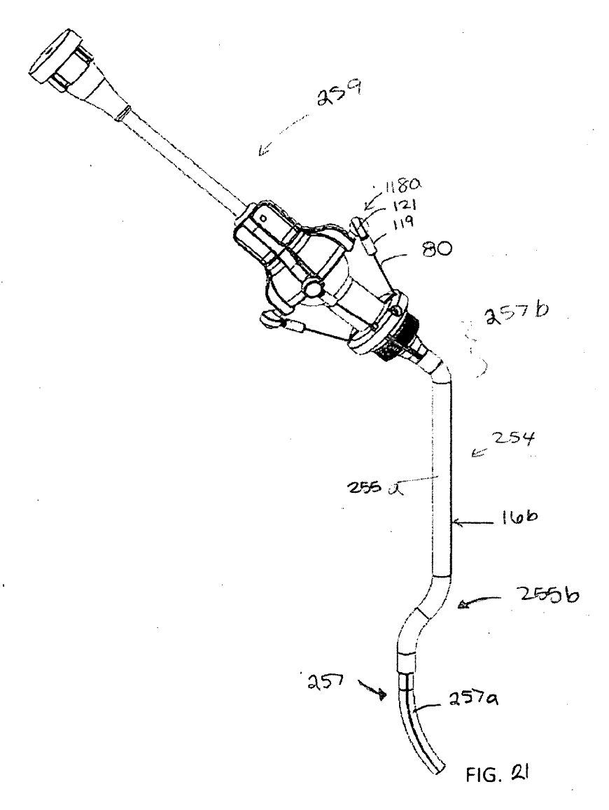

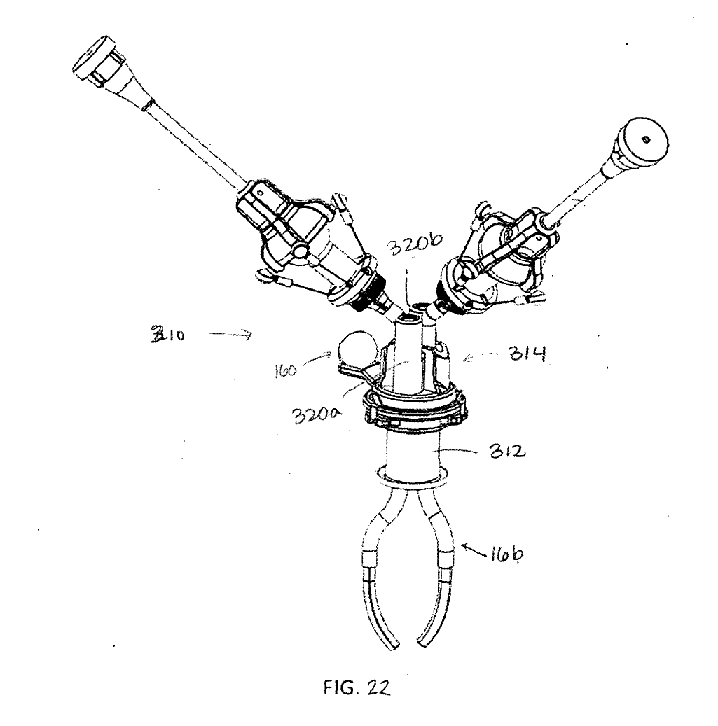

Referring to FIG. 22, the system 310 of the third embodiment includes a base 312 and an upper housing 314. The features of the base 312 may be similar to those described in connection with the first embodiment, as shown in FIGS. 23 and 24.

The proximal section of each rigid tube 354 is moveably coupled to the upper housing 314. As with the first and second embodiments, active ports in the form of deflectable instrument delivery tubes 16b are supported by the upper housing 314. The instrument delivery tubes 16b and associated actuators share many features with those of the first embodiment, including rigid tubes 354 and flexible tubes 357 extending through the rigid tubes 354. However, in the instrument delivery tubes of the second embodiment, the rigid tubes 354 extend fully to the actuator rather than leaving an exposed portion of the flexible tube 357 as was shown in FIG. 21.

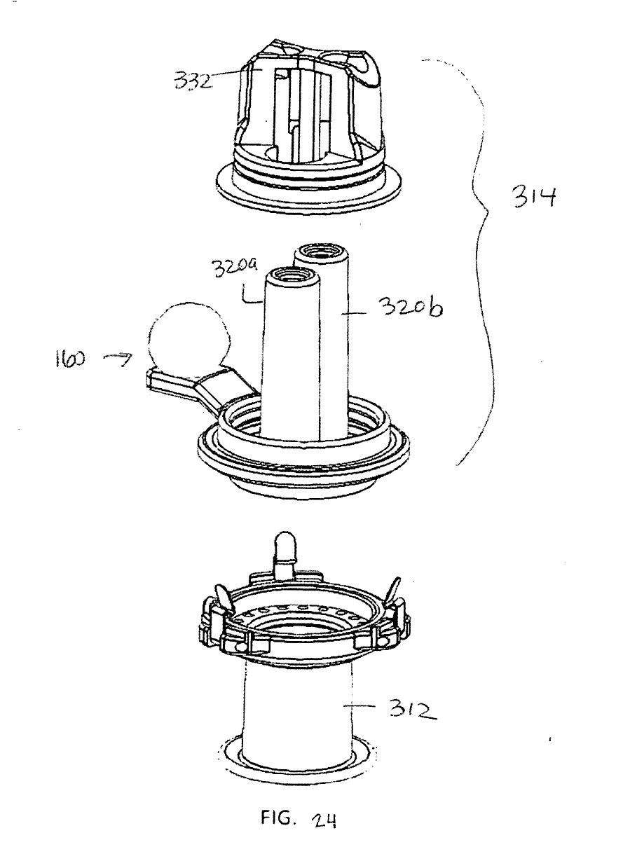

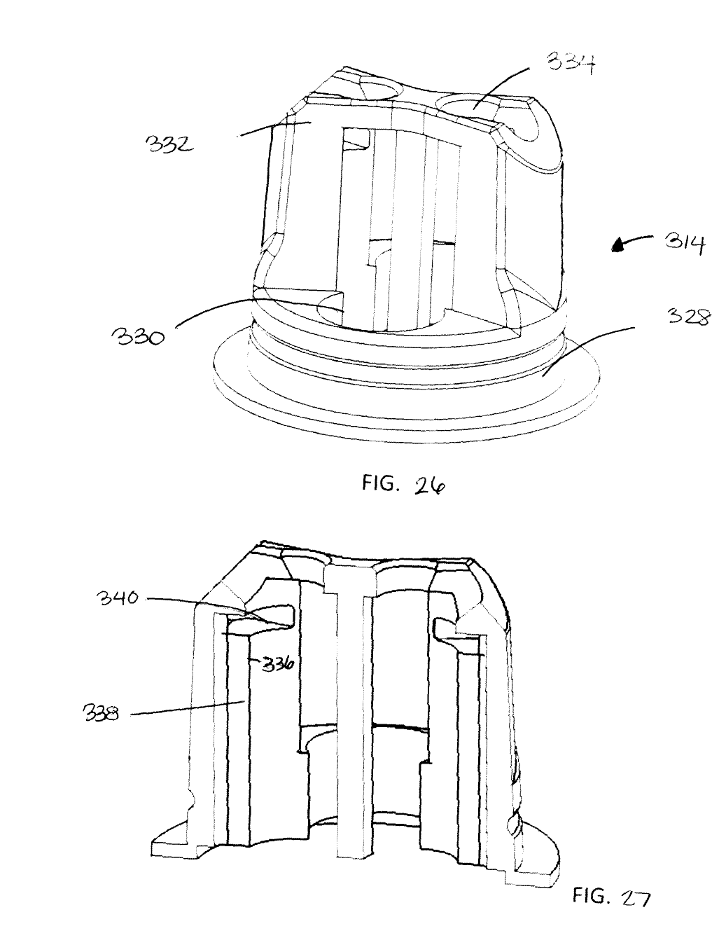

Referring to FIG. 26, the upper housing 314 includes a lower plate section 328 having individual or interconnecting openings 330. The instrument delivery tubes 16b extend through of the openings 330. Rigid, proximally-extending support members 332 extend from the lower plate section as shown. The members 332 are shaped to receive and rigidly support the proximal portions of the rigid tubes as shown in FIG. 28, and to prevent pivotal movement of the rigid tubes 354. The members may be tubular, or they might have a partially tubular or open construction as shown. In the illustrated embodiment, each member 332 includes an opening 334 through which an instrument delivery tube may be inserted. Each member includes an inner surface having a guide slot 336 with a longitudinal portion 338 and a circumferential portion 340.

A bushing 342 mounted to the shaft of each stiffening tube 354 includes a protrusion 346 that extends into the L-shaped slot 336. The position of the protrusion relative to each stiffening tube is such that when the stiffening tubes are in the closed position (as in FIG. 15A), the protrusion is positioned within the circumferential portion of the guide slot 336, away from the longitudinal portion. To axially rotate the instrument delivery tubes to the deployed positions, the user will rotate the rigid tubes 354, causing them to axially rotate. When the rigid tubes have been rotated sufficiently to position the protrusion of the bushing into alignment with the longitudinal portion of the guide slot the instrument delivery tubes may be longitudinally advanced further into the body if desired. The longitudinal position of the instrument delivery tubes may be altered during the course of the procedure in this manner.

A pair of tubular ports 320a, 320b extend from the upper housing section 314 and through two of the openings 330 in the lower plate section 328. The ports 320a, 320b are passive ports for receiving instruments to be inserted into the body cavity. These ports may take the form of detachable ports each of which might have a duckbill valve and annular instrument seal similar to those described above in connection with the second embodiment. The ports 320a, 320b may be of equal size, or the sizes may differ between the ports.

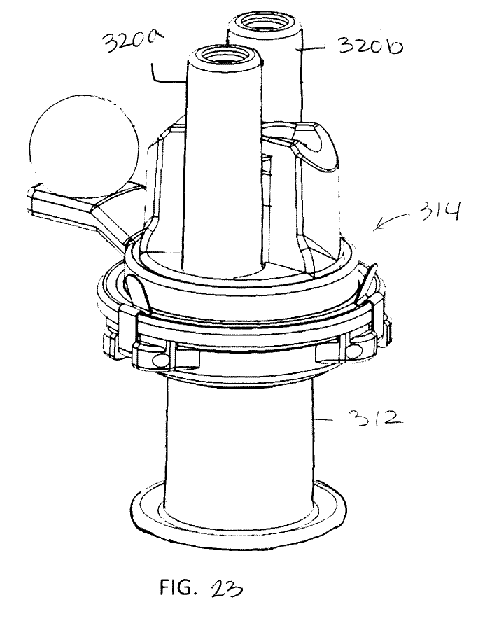

Referring to FIG. 25, the distal end of each port 320a, 320b includes a circumferential groove 318 proximally offset from the distal end of the port. A plate 324 disposed within the system, such as on the ledge 329 discussed in the first embodiment (FIG. 18), includes openings 326 for receiving the ports. To mount a port 320a to the plate, the distal end of the port is inserted into one of the openings. The port is pressed downwardly to cause groove 318 to contact the portion of the wall surrounding the opening in the plate, thereby forming a seal around the opening. It should be noted that the other openings 328 in the plate are positioned so that the instrument delivery tubes may extend through them.

Referring to FIGS. 23 and 24, the spherical mount 160 is positioned on a collar that is rotatably positioned on the base or upper housing, allowing the entire system to be axially rotated relative to the mount if repositioning is needed.

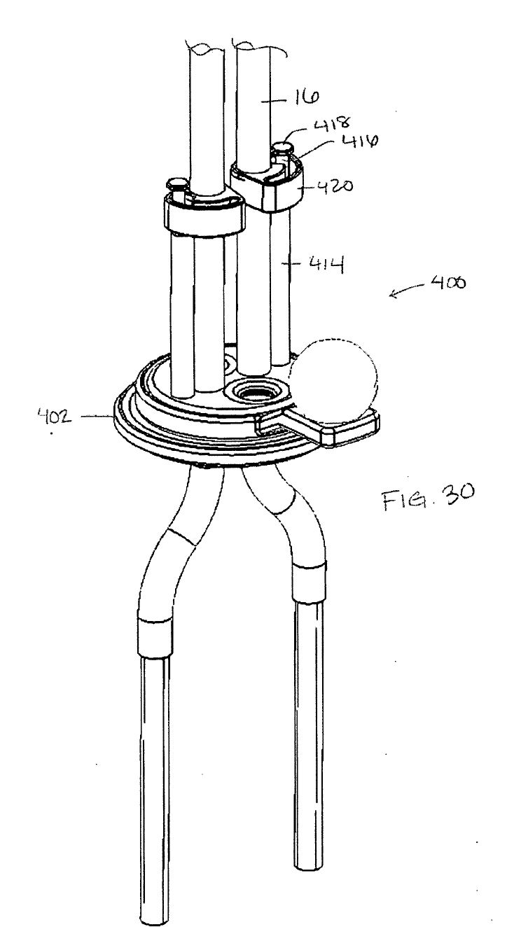

A fourth embodiment of an access system 400 is shown in FIG. 30. The access system 400 is similar to that of the third embodiment in that it is designed to restrict or prevent longitudinal movement of the instrument delivery tubes when they are in the closed position (e.g. similar to that shown in FIG. 9A), and to allow longitudinal movement once the instrument delivery tubes have been axially rotated into the deployed position such as that shown in FIG. 30. As with the first through third embodiments, the instrument delivery tubes are restricted against pivotal movement relative to the main access cannula or base.

System 400 includes a proximal housing 402 which may be coupled or attachable to a distal housing or cannula positionable in an incision. The distal housing may be similar to that of any of the previously described embodiments (e.g. main tube 12 of FIG. 1 or base 212 of FIG. 15).

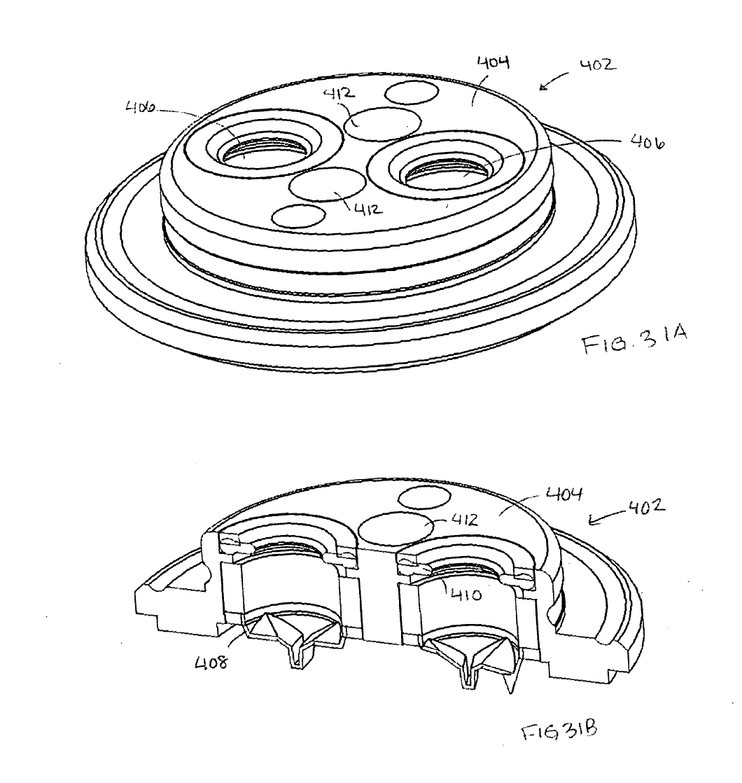

Referring to FIG. 31, the proximal housing 402 includes a proximal surface 404. A pair of bores 406 extend through the housing 402 from the proximal surface 404. The bores 406 function as access ports for instruments to be used in the body cavity.

As shown in FIG. 31B, each bore includes a valve 408 such as a cross-slit or duck bill valve recessed below the surface 404. The valves 408 function to seal the bores during times when the bores are not occupied by instruments. Septum seals 410 are positioned proximal to the valves 408 and serve to seal against the shafts of instruments passed through the ports.

Two additional bores 412 extend through the proximal housing 402. As shown in FIG. 30, instrument delivery tubes 16 are disposed in the bores 412. The instrument delivery tubes 16 may be similar to those described in connection with the first, second and third embodiments, or alternate instrument delivery tubes may instead be used.

Posts 414 extend proximally from the surface 404, in parallel to the instrument delivery tubes. Each post includes a distal section 415, a reduced diameter section 416, and a proximal head 418 that is broader than the reduced diameter section 416.

Guides 420 are mounted to the shaft of each instrument delivery tube 16. Each guide 420 includes a cutout 422 extending through the guide in the longitudinal direction. The cutout curves in parallel to the cylindrical outer surface of the instrument delivery tube. The cutout has a sort of “apostrophe” shape, with a main section 424 and an enlarged generally cylindrical section 426 is positioned at one end of the main section 424. The radial width of the main section 424 is narrower than the diameter of the head 418 or the distal section 415 of the post 414, whereas the enlarged section 426 is shaped and sized to allow the head 418 and distal section 415 to pass through.

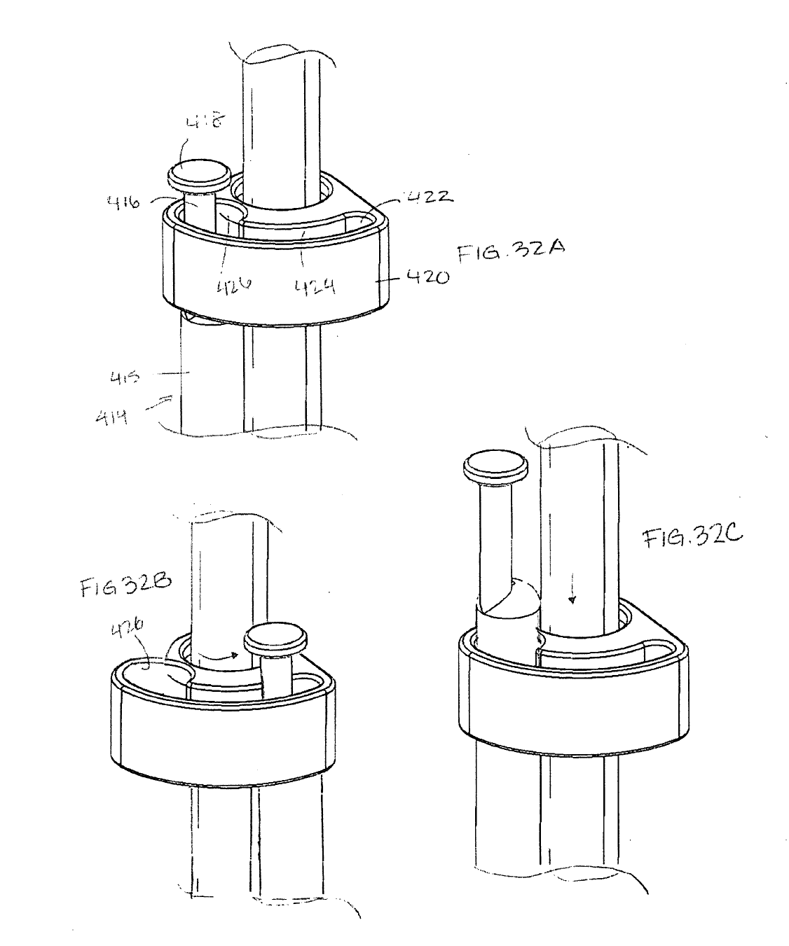

As with the prior embodiments, the instrument delivery tubes 16 are axially rotatable. Axial rotation of the instrument delivery tubes 16 likewise rotates the guides 420, thus changing their positions relative to the posts 414. When an instrument delivery tube 16 is axially positioned such that the longitudinal axis of the guide's enlarged cutout section 426 is aligned with the longitudinal axis of the post 414 (see FIG. 32A), the distal portion 66 of the instrument delivery tube is in the fully deployed position shown in FIG. 30. When an instrument delivery tube is in the deployed position, the enlarged section 426 of the cutout in the guide 420 is axially aligned with the post 414, allowing for longitudinal movement of the instrument delivery tube as illustrated in FIG. 32C since the enlarged section 426 is sufficiently large to slide over the head 418 and distal section 415 of the post 414.

The instrument delivery tube 16 may be axially rotated towards the closed position when the reduced diameter section 416 of the post 414 is disposed within the cutout 422. Axial rotation of the instrument delivery tube 16 such that the end of the cutout 424 opposite from the enlarged section 426 receives the post 414 as is shown in FIG. 32B places the distal portions 66 of the instrument delivery tubes in a closed position similar to that shown in FIG. 9A. Note that when the longitudinal axis of the enlarged section of the cutout 426 is axially offset from the longitudinal axis of the post 414 as in FIG. 32A, the head 418 and distal section 415 of the post 414 limit or prevent longitudinal movement of the instrument delivery tube since they cannot pass through the main section 424 of the cutout. Thus, in the preferred embodiment, when the instrument delivery tubes are in the closed position, they are restricted against longitudinal movement.

While certain embodiments have been described above, it should be understood that these embodiments are presented by way of example, and not limitation. It will be apparent to persons skilled in the relevant art that various changes in form and detail may be made therein without departing from the spirit and scope of the invention. This is especially true in light of technology and terms within the relevant art(s) that may be later developed.

Any and all patents, patent applications and printed publications referred to above, including for purposes of priority, are incorporated herein by reference.

1. A surgical access system comprising:
a sealed base positionable in an incision formed in a body wall;
at least two access tubes extending through the base, each access tube including
a rigid tube extending through the base and having a proximal section, and a distal section positioned distally of the base, the rigid tube having a fixed pre-formed shape including a bend in the distal section, each rigid tube restrained against pivotable movement relative to the base; and
a deflectable tube extending from the distal end of the rigid tube and including a lumen for passage of a medical instrument therethrough.

2. The system of claim 1, wherein the proximal section of each rigid tube includes a straight section at least partially disposed within the base, and the rigid tubes are oriented such that the straight sections extend and are fixed in parallel to one another.

3. The system of claim 1, wherein at least one of the rigid tubes is axially rotatable within the base relative to a longitudinal axis of the straight section.

4. The system of claim 3, wherein the axially rotatable rigid tube is axially rotatable between predetermined first and second axial positions, the rigid tube retainable in each of the predetermined first and second axial positions.

5. The system of claim 4, wherein the system includes a first element coupled to the axially rotatable rigid tube and a second element coupled to the base, wherein the first and second elements are engageable when the rigid tube is in the first position to retain the rigid tube in the first position, and wherein the first and second elements are engageable when the rigid tube is in the second position to retain the rigid tube in the second position.

6. The system of claim 5 wherein the second element includes a first slot and a second slot, and wherein the first element is insertable into the first slot to retain the rigid tube in the first position, and wherein the first element is insertable into the second slot to retain the rigid tube in the second position.

7. The system of claim 6, wherein the first element is longitudinally advanceable and retractable within the first slot to adjust a longitudinal position of the rigid tube relative to the base.

8. The system of claim 7, wherein the first slot includes a plurality of longitudinally spaced catch features, the first element selectively engageable with the catch features to retain the rigid tube in a select ones of a plurality of predetermined longitudinal positions.

9. The system of claim 1 wherein the rigid tube is selectively retainable in a plurality of predetermined longitudinal positions.

10. The system of claim 1 wherein the deflectable tubes have a fixed longitudinal position relative to the rigid tubes.

11. The system of claim 1, further including a proximal element coupled to the base, wherein the proximal section of each rigid tube is coupled to the proximal element and wherein the proximal element restrains the rigid tubes against pivotable movement.

12. The system of claim 11 wherein the proximal element comprises a proximal housing, and wherein the proximal sections of the rigid tubes are coupled to the housing.

13. The system of claim 11, wherein each proximal element includes a post extending proximally from the base.

14. The system of claim 1, wherein the base is a tubular cannula having a lumen, wherein the rigid tubes extend through the lumen, and wherein the system further includes a restraint coupled to the base and positioned in contact with the distal sections of the rigid tubes to prevent pivotable movement thereof.

15. The system of claim 14 wherein the restraint includes a partition having at least two holes therein, the distal sections of the rigid tubes extending through the holes in the partition.

16. The system of claim 1, wherein each deflectable tube is operatively associated with an elongate actuation element and an actuator having first and second actuator portions, a distal portion of the actuation element coupled to the deflectable tube and a proximal portion of the actuation element coupled to the second actuator portion, wherein the first actuator portion is positioned on the proximal section of a corresponding rigid tube and the second actuator portion is moveably coupled to the first portion and positioned such that when an instrument is disposed in the lumen of the rigid tube, a portion of the instrument's handle contacts the second portion such that pivotal movement of the instrument's handles moves the second portion actuator relative to the first actuator portion to activate the elongate actuation element.

17. The system of claim 16, wherein each deflectable tube includes a plurality of actuation elements.

18. The system of claim 1, wherein each deflectable tube includes a distal end positioned distal to the distal end of the corresponding rigid tube and a proximal section disposed within the proximal section of the corresponding rigid tube.

19. The system of claim 1, wherein each deflectable tube is in a fixed longitudinal position relative to its corresponding rigid tube.

20. The system of claim 3, wherein the at least one rigid tube is axially rotatable between a first position in which the bends of the distal sections bend in parallel to one another, and a second position in which at least portions of the bends of the distal sections curve or angle away from one another.

21. The system of claim 3 wherein the at least one rigid tube is axially rotatable between a first position in which the maximum separation distance between the distal sections has a first length and a second position in which the maximum separation distance between the distal sections has a second length longer than the first length.

22. The system of claim 1, further including a support arm attachable to the base and to a patient treatment table.

23. The system of claim 1, further including at least one secondary tube extending through the base in parallel to the rigid tubes, the secondary tube having a distal end disposed within the base and a proximal end positioned proximal to the base, the proximal end including a sealed port.

24. The system of claim 1, further including seals sealing the system against proximal movement of gas via the access tubes and out of the system.

25. The system of claim 1, wherein the base comprises a tube having at least one seal positioned to seal a lumen of the tube.

26. The system of claim 25, wherein the access tubes extend proximally through the seal.

27-47. (canceled)
